(19) United States
(12) Patent Application Publication
(10) Pub. No.: US 2026/0041370 A1
(43) Pub. Date: Feb. 12, 2026

(54) ANXIETY MAP DISPLAY DEVICE AND ANXIETY FEATURE PRESENTATION DEVICE

(71) Applicant: Panasonic Automotive Systems Co., Ltd., Kanagawa (JP)

(72) Inventors: Tetsuo MATSUSE, Osaka Fu (JP); Motoyuki OKAYAMA, Osaka Fu (JP); Tatsuya KOYAMA, Kyoto Fu (JP); Koji ISHII, Kanagawa Ken (JP); Shin HIRANO, Osaka Fu (JP); Yukihiro MORITA, Osaka Fu (JP)

(73) Assignee: Panasonic Automotive Systems Co., Ltd., Kanagawa (JP)

(21) Appl. No.: 19/272,571

(22) Filed: Jul. 17, 2025

(30) Foreign Application Priority Data
Aug. 9, 2024 (JP) 2024-134164

(51) Int. Cl. A61B 5/00; B60Q 9/00; G06T 11/00; G06T 11/20; G07C 5/04; G09B 19/00

(52) U.S. Cl. CPC A61B 5/4884; A61B 5/742; B60Q 9/00; G06T 11/001; G06T 11/206; G07C 5/04; G09B 19/00

(57) ABSTRACT

An anxiety map display device according to the present disclosure includes a memory, and a processor coupled to the memory. The processor is configured to: acquire an anxiety state of a driver during driving; acquire position information of a vehicle during driving; acquire a driving situation of the vehicle during driving; generate an anxiety map indicating an anxiety occurrence position corresponding to a predetermined driving situation for the driver based on the position information and the driving situation when acquiring a predetermined anxiety state for the driver; and display the generated anxiety map for the driver as an individual anxiety map.

### CROSS-REFERENCE TO RELATED APPLICATIONS

This application is based upon and claims the benefit of priority from Japanese Patent Application No. 2024-134164, filed on Aug. 9, 2024, the entire contents of which are incorporated herein by reference.

### FIELD

The present disclosure relates to an anxiety map display device and an anxiety feature presentation device.

### BACKGROUND

Conventionally, an anxiety map has been proposed in which a place where anxiety is likely to occur during driving is displayed on a map.

By effectively using this anxiety map, it becomes possible for the driver to drive with greater peace of mind, and by using the anxiety map to provide driving assistance, more effective driving assistance can be achieved.

Related techniques are described in JP 2022-109551 A, and JP 2020-148613 A

However, the above conventional anxiety map is not displayed in accordance with the anxiety characteristics (a situation where anxiety is felt, a driving situation where anxiety is likely to be felt, and the like) of the driver who actually drives, and is merely displayed in accordance with the anxiety characteristics of general drivers.

Therefore, it does not necessarily follow the sense of each driver, and there is a possibility that the anxiety map cannot be effectively used.

Similarly, even when the driving assistance parameter control is performed based on the generated anxiety map, it is not always possible to perform effective driving assistance for each driver.

In view of the above problems, an object of the present disclosure is to provide an anxiety map display device and an anxiety feature presentation device capable of providing information more suitable for each driver based on the anxiety characteristics of each driver, thereby enabling driving with greater peace of mind.

### SUMMARY

In order to achieve the object, an anxiety map display device according to the present disclosure includes a memory, and a processor coupled to the memory. The processor is configured to: acquire an anxiety state of a driver during driving; acquire position information of a vehicle during driving; acquire a driving situation of the vehicle during driving; generate an anxiety map indicating an anxiety occurrence position corresponding to a predetermined driving situation for the driver based on the position information and the driving situation when acquiring a predetermined anxiety state for the driver; and display the generated anxiety map for the driver as an individual anxiety map.

### BRIEF DESCRIPTION OF THE DRAWINGS

FIG. 1 is a functional configuration block diagram of an anxiety map display device according to a first embodiment;

FIG. 2 is an explanatory diagram of an example of acquisition of anxiety state information by an anxiety state acquisition unit in the anxiety map display device according to the embodiment;

FIG. 3 is a processing flowchart of information accumulation processing of accumulating anxiety occurrence time information according to the embodiment;

FIG. 4 is a processing flowchart (part 1) of an anxiety map display processing;

FIG. 5 is a processing flowchart (part 2) of anxiety map display;

FIG. 6 is a processing flowchart (part 3) of the anxiety map display;

FIGS. 7A and 7B are explanatory diagrams of a display example in a case where an occurrence frequency image is superimposed and displayed on a map image;

FIG. 8 is a functional configuration block diagram of an anxiety map display system according to a second embodiment;

FIG. 9 is an operation processing flowchart at the time of acquiring driver characteristics and attributes;

FIG. 10 is an operation processing flowchart at the time of information accumulation;

FIG. 11 is a processing flowchart of accumulated information transmission processing;

FIG. 12 is an explanatory diagram of a display processing sequence of a general anxiety map;

FIG. 13 is an explanatory diagram of a modification example of the display processing sequence of the general anxiety map;

FIG. 14 is a processing flowchart of information accumulation processing of accumulating anxiety occurrence time information according to a third embodiment;

FIG. 15 is an explanatory diagram of an example of anxiety occurrence time information data;

FIG. 16 is an explanatory diagram of an anxiety state section link list;

FIG. 17 is an explanatory diagram of a data format of condition data output by a condition data acquisition unit;

FIG. 18 is an explanatory diagram of an example of a condition acquisition screen in a case where the general anxiety map is displayed;

FIG. 19 is an explanatory diagram of a setting screen for characteristics and attributes of a person in question;

FIGS. 20A and 20B are explanatory diagrams of a derivation method in a case of deriving the anxiety characteristics of a user from the user's responses to questions;

FIG. 21 is a functional configuration block diagram of anxiety feature quantities presentation device;

FIG. 22 is a diagram for describing an example of calculation results of anxiety feature quantities for each driving scene;

FIG. 23 is a diagram for describing an example of calculation results of anxiety feature quantities for each driving environment;

FIG. 24 is a diagram for describing an example of calculation results of anxiety feature quantities for each driving scene in which anxiety is generally less likely to occur;

FIG. 25 is a diagram for describing an example of calculation results of anxiety feature quantities for each case of presence or absence of a passenger;

FIG. 26 is a diagram for describing an example of calculation results of anxiety feature quantities for each first-time-driving road;

FIG. 27 is a diagram illustrating an example of calculation results of anxiety feature quantities for each predetermined period immediately after the start of driving;

FIG. 28 is a processing flowchart of processing of calculating anxiety feature quantities for each driving scene;

FIG. 29 is a processing flowchart of processing of calculating anxiety feature quantities for each driving environment;

FIG. 30 is a processing flowchart of processing of calculating anxiety feature quantities for each driving scene where anxiety is generally less likely to occur;

FIG. 31 is a processing flowchart of processing of calculating anxiety feature quantities on the first-time-driving road;

FIG. 32 is a processing flowchart of processing of calculating anxiety feature quantities in a predetermined period immediately after the start of driving;

FIG. 33 is a processing flowchart of processing of calculating anxiety feature quantities in the presence or absence of a passenger;

FIG. 34 is a functional configuration block diagram of an information presentation device according to a first aspect of a fifth embodiment;

FIG. 35 is a functional configuration block diagram of an information presentation device according to a second aspect of the fifth embodiment;

FIG. 36 is a functional configuration block diagram of an information presentation device according to a third aspect of the fifth embodiment;

FIG. 37 is an explanatory diagram (part 1) of comparison data of anxiety feature quantities for each driving scene;

FIG. 38 is an explanatory diagram (part 2) of comparison data of anxiety feature quantities for each driving scene;

FIG. 39 is an explanatory diagram (part 3) of comparison data of anxiety feature quantities for each driving scene;

FIG. 40 is an explanatory diagram of an example of advice corresponding to a combination of driving skill and changes in anxiety feature;

FIG. 41 is an operation flowchart of an information presentation device 80 according to the third aspect; and

FIG. 42 is a processing flowchart of determination processing of an anxiety feature change determination unit.

### DETAILED DESCRIPTION

Next, embodiments will be described in detail with reference to the drawings.

### (1) First Embodiment

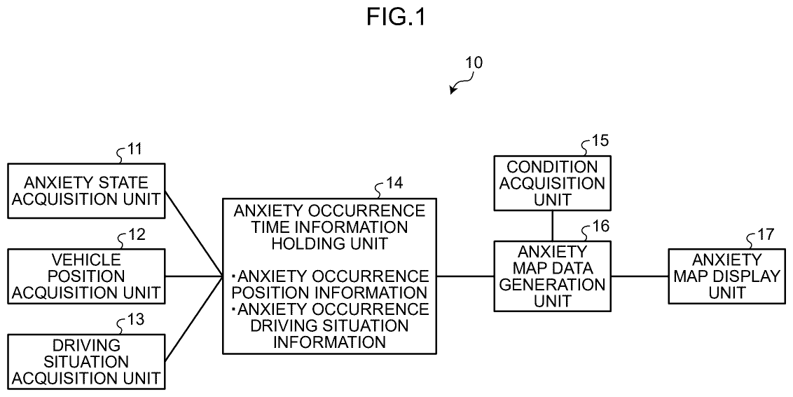

FIG. 1 is a functional configuration block diagram of an anxiety map display device according to a first embodiment.

An anxiety map display device 10 includes an anxiety state acquisition unit 11, a vehicle position acquisition unit 12, a driving situation acquisition unit 13, an anxiety occurrence time information holding unit 14, a condition acquisition unit 15, an anxiety map data generation unit 16, and an anxiety map display unit 17.

The anxiety state acquisition unit 11 acquires anxiety state information indicating an anxiety state of a driver of a vehicle.

The anxiety state acquisition unit 11 acquires, for example, biological information such as heart rate of the occupant or electro dermal activity (EDA) of the occupant, and acquires the anxiety state based on the acquired biological information.

For details of the acquisition of the anxiety state based on the biological information, refer to, for example, “Kiba and Osuga, ‘Development of an Anxiety Level Estimation Technique Using Physiological Index for VR Exposure Therapy System’, Journal of Biomedical Engineering, 2020”.

More specifically, the anxiety state acquisition unit 11 sequentially acquires, for example, information indicating the heart rate of the occupant and information indicating EDA from an electrode attached to the steering wheel.

Note that the heart rate and EDA of the occupant can also be acquired from a wearable device worn on a wrist, an arm, a head, or the like of the occupant, for example, a smartwatch or the like.

In addition, as an example of an algorithm for acquiring the anxiety state, the anxiety state acquisition unit 11 acquires the anxiety state information indicating that the driver is in the anxiety state in a case where an average value of R-R interval (RRI), which is acquired heart rate variability time-series data, decreases and an average value of skin conductance (SC) which is one piece of information of the acquired EDA greatly increases.

Note that, as a method for acquiring the anxiety state, in addition to the method for automatically detecting the anxiety state by the biological information (the above example) or the like, the voice uttered by the user, who is the driver, indicating an anxiety state may be recognized, and it may be recognized that the user is in an anxiety state, or a self-reported utterance of anxiety may be acquired by pressing a button.

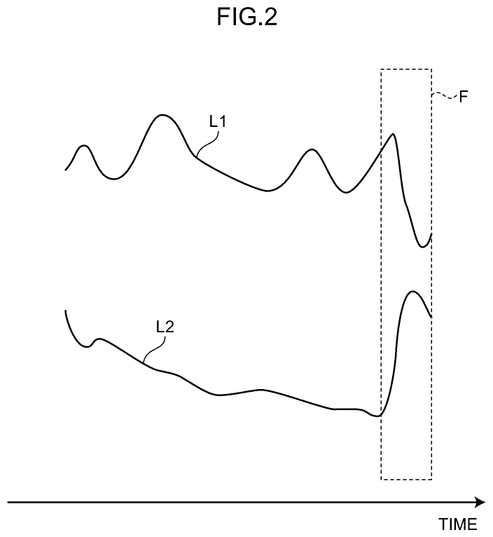

FIG. 2 is an explanatory diagram of an example of acquisition of anxiety state information by an anxiety state acquisition unit in the anxiety map display device according to the embodiment.

In FIG. 2, a solid line L1 represents a time-series change in an average value of RRI of the occupant, and a solid line L2 represents a time-series change in an average value of SC of EDA.

In FIG. 2, in the time zone surrounded by the broken line, the average value of RRI corresponding to the solid line L1 decreases, and the average value of SC of EDA corresponding to the solid line L2 greatly increases.

Therefore, in the example illustrated in FIG. 2, the anxiety state acquisition unit 11 acquires the anxiety state information indicating that the driver is in the anxiety state in the time zone surrounded by the broken-line frame F.

The vehicle position acquisition unit 12 identifies the own vehicle position by satellite positioning by a global navigation satellite system (GNSS), autonomous positioning using an acceleration sensor, a gyro sensor, or the like, map matching, or the like.

The driving situation acquisition unit 13 acquires the driving situation of the own vehicle based on the route search result, the map matching result, and the like. Here, the driving situation is a driving situation that may cause an anxiety state such as a width, a lane, a type of a road (an automobile-only road including a highway or a general road) on which the own vehicle is traveling, a right turn, a lane change, merging, parking, traffic congestion information provided by a traffic control system, nighttime driving acquired from time information, and rainy weather driving detected by wiper operation or sensors, and is not limited thereto.

When the anxiety state is detected in the anxiety state acquisition unit 11, the anxiety occurrence time information holding unit 14 stores the vehicle position acquired by the vehicle position acquisition unit, the driving situation acquired by the driving situation acquisition unit, and the date and time information in association with each other.

The condition acquisition unit 15 receives input from the user regarding the conditions for generating the anxiety map.

As the condition, for example, a traffic condition (for example, a narrow road, a right turn, a highway, or the like) in which the user is likely to feel anxiety is specified.

The anxiety map data generation unit 16 refers to the anxiety occurrence time information holding unit 14, extracts a place where the user or the general driver is likely to feel anxiety on the map screen based on the condition acquired by the condition acquisition unit 15 and the route of the navigation target, generates the anxiety map data for displaying the anxiety map (the individual anxiety map or the general anxiety map), and outputs the anxiety map data to the anxiety map display unit 17.

The anxiety map display unit 17 is configured as, for example, a display, and displays an individual anxiety map specialized for the user or a general anxiety map considered to be applicable to a general driver based on the anxiety map data generated by the anxiety map data generation unit 16.

Next, an operation of the embodiment will be described.

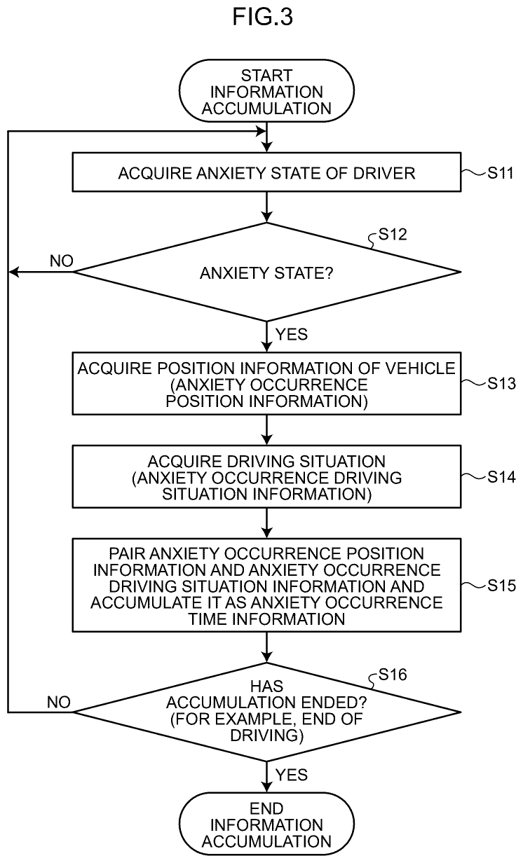

FIG. 3 is a processing flowchart of information accumulation processing of accumulating anxiety occurrence time information according to the embodiment.

First, the anxiety state acquisition unit 11 of the anxiety map display device 10 starts an operation of acquiring anxiety state information indicating the anxiety state of the driver of the vehicle (step S11).

As a result, the anxiety occurrence time information holding unit 14 determines whether or not the anxiety state is acquired in the anxiety state acquisition unit 11 (step S12).

In the determination of step S12, when the anxiety state has not been acquired yet (step S12; No), the process proceeds to step S11 and enters a standby state.

In the determination of step S12, when the anxiety state is acquired (step S12; Yes), the anxiety occurrence time information holding unit 14 acquires the anxiety occurrence position information which is the position information of the own vehicle from the vehicle position acquisition unit 12 (step S13).

Further, the anxiety occurrence time information holding unit 14 acquires the anxiety occurrence driving situation information which is the driving situation of the own vehicle from the driving situation acquisition unit 13 (step S14).

Subsequently, the anxiety occurrence time information holding unit 14 pairs the acquired anxiety occurrence position information and the anxiety occurrence driving situation information and accumulates the information as the anxiety occurrence time information together with the date and time information (step S15).

Then, the anxiety occurrence time information holding unit 14 determines whether or not the accumulation end timing of anxiety occurrence time information has been reached, for example, whether or not the driving has ended (step S16).

In the determination of step S16, when the accumulation end timing of anxiety occurrence time information has not been reached (step S16; No), the processing proceeds to step S11 again, and thereafter, the above-described processing is executed.

In the determination of step S16, when the accumulation end timing of the anxiety occurrence time information has been reached (step S16; Yes), the anxiety occurrence time information holding unit 14 ends the processing.

Next, an operation when the anxiety map is displayed will be described.

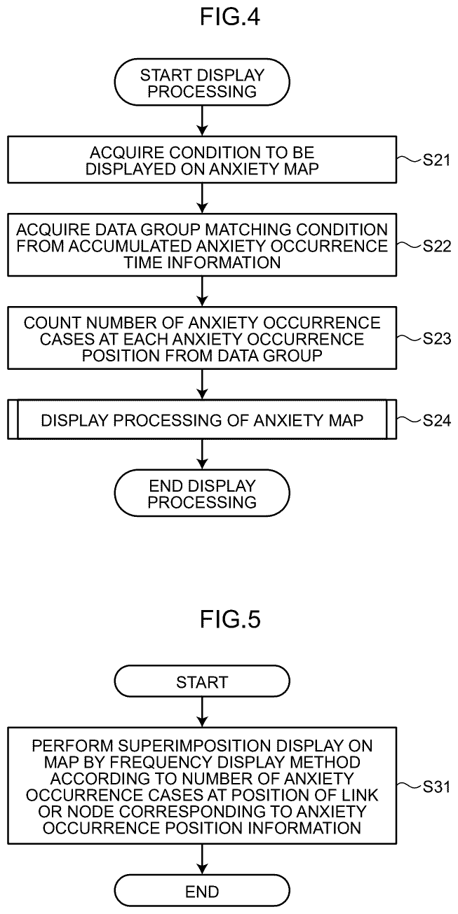

FIG. 4 is a processing flowchart (part 1) of an anxiety map display processing.

In this case, it is assumed that the user inputs a condition of information to be displayed in the anxiety map prior to the anxiety map display.

Note that a default display information condition can also be used as the condition of the information displayed on the anxiety map.

As a result, the condition acquisition unit 15 acquires a condition (user input information or default information) for generating the anxiety map, and outputs the acquired condition to the anxiety map data generation unit 16 (step S21).

As a result, the anxiety map data generation unit 16 to which the condition for generating the anxiety map is input acquires a plurality of pieces of data (data group) matching the condition from the anxiety occurrence time information accumulated in the anxiety occurrence time information holding unit 14 (step S22).

Subsequently, the anxiety map data generation unit 16 counts the number of anxiety occurrence cases at each anxiety occurrence position from the acquired data group (step S23).

Then, the anxiety map data generation unit 16 outputs the generated anxiety map data to the anxiety map display unit 17 to perform the display processing of the anxiety map (step S24).

FIG. 5 is a processing flowchart (part 2) of the anxiety map display.

Based on the anxiety map data generated by the anxiety map data generation unit 16, the anxiety map display unit 17 superimposes and displays the occurrence frequency image on the map image by the frequency display method according to the number of anxiety occurrence cases at the (display) position of the link or node corresponding to the anxiety occurrence position information (step S31).

In this case, it is preferable to display the occurrence frequency image in a display color that does not exacerbate anxiety.

Specifically, the display color that does not exacerbate anxiety is a display color that gives an impression of sedation and relaxation and is likely to activate the parasympathetic nerve, for example, blue and green are used as main colors.

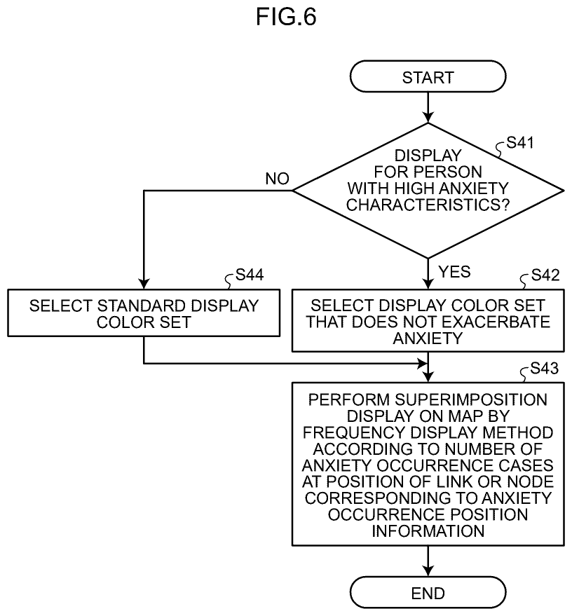

FIG. 6 is a processing flowchart (part 3) of the anxiety map display.

The display processing of the anxiety map of FIG. 6 is different from the display processing of the anxiety map of FIG. 5 in that the user can select a display color for displaying the occurrence frequency image.

First, the anxiety map display unit 17 determines whether or not display for a person with high anxiety characteristics is specified based on the anxiety map data generated by the anxiety map data generation unit 16 (step S41).

In the determination of step S41, when the display for a person with high anxiety characteristics is specified (step S41; Yes), the anxiety map display unit 17 selects a display color set that does not exacerbate anxiety (step S42).

Specifically, the display color set that does not exacerbate anxiety is a display color set that gives an impression of sedation and relaxation and is likely to activate the parasympathetic nerve, for example, a display color set using blue and green as main colors.

In the determination of step S31, when the display for a person with high anxiety characteristics is not specified (step S41; No), the anxiety map display unit 17 selects a standard display color set (step S42).

Specifically, it is a display color set in which red and yellow are used as main colors to further raise attention as in the related art.

Subsequently, the anxiety map display unit 17 performs superimposition display on the map by a frequency display method according to the number of anxiety occurrence cases at the position of the link or the node corresponding to the anxiety occurrence position information (step S43).

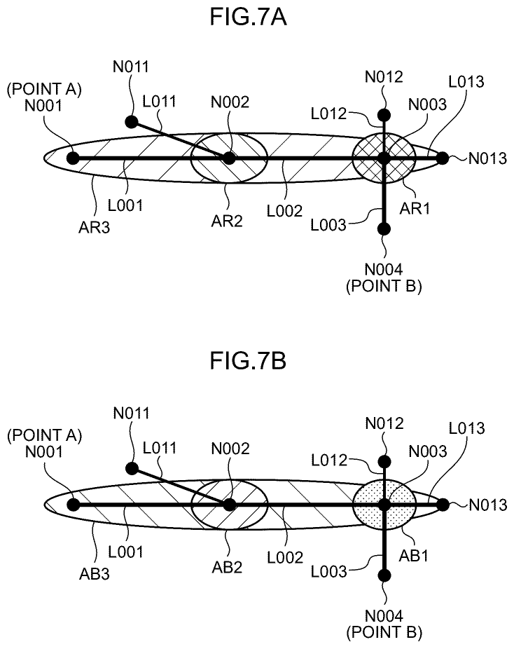

FIGS. 7A and 7B are explanatory diagrams of a display example in a case where the occurrence frequency image is superimposed and displayed on the map image.

In FIGS. 7A and 7B, for easy understanding, nodes and links as map data corresponding to a map image of a road are displayed instead of a map image of a road.

In the example of FIGS. 7A and 7B, a case of moving from a point A to a point B is assumed.

On the map data, the point A corresponds to the node N001, and the point B corresponds to the node N004.

Further, the shortest route from the point A to the point B can be represented by node N001→link L001→node N002→link L002→node N003→link L003→node N004.

In this case, it is assumed that the occurrence frequency of anxiety is divided into three frequencies of a first occurrence frequency at which the occurrence frequency of anxiety is relatively low, a second occurrence frequency at which the occurrence frequency of anxiety is higher than the first occurrence frequency, and a third occurrence frequency at which the occurrence frequency of anxiety is relatively high.

First, a case where the occurrence frequency of anxiety is superimposed and displayed on a map in a standard display color set will be described with reference to FIG. 7A.

In the case of the example of FIG. 7A, the route to node N001→link L001→node N002→link L002→node N003→link L013→node N013 is, for example, a route corresponding to the first occurrence frequency in which the occurrence frequency of anxiety is relatively low although the route is a section in which the road width is narrow and the user feels some anxiety. Note that the route in which the occurrence frequency of anxiety is equal to or less than the predetermined threshold is not displayed in a superimposed manner.

In this manner, in the route in which the occurrence frequency of anxiety corresponds to the first occurrence frequency, for example, a yellow occurrence frequency image AR3 is superimposed and displayed on the map in the standard display color set.

In addition, the node N002 is a merging point where the road of the link L011 merges with the road of the link L001, and the occurrence frequency of anxiety is the second occurrence frequency higher than the first occurrence frequency.

In this manner, in the route in which the occurrence frequency of anxiety corresponds to the second occurrence frequency, for example, a vermilion occurrence frequency image AR2 is superimposed and displayed on the map in the standard display color set.

In addition, when the node N003 is viewed from the vehicle traveling on the road of the link L002, traveling that changes the direction with respect to the link L003 corresponds to an intersection where the vehicle turns right, and the occurrence frequency of anxiety is a third occurrence frequency that is relatively high.

In this manner, in the route in which the occurrence frequency of anxiety corresponds to the third occurrence frequency, for example, a vermilion occurrence frequency image AR1 is superimposed and displayed on the map using the standard display color set, thereby providing a display that prompts maximum caution.

As described above, according to the first embodiment, since the anxiety map reflecting the characteristics and attributes of the specific user (driver) can be displayed and used, it becomes possible to drive with a margin using the user-specific anxiety map based on the driving history up to that point.

### (2) Second Embodiment

While the above first embodiment is an anxiety map display device adapted to a specific user, the second embodiment is an anxiety map display system adapted to an unspecified large number of users.

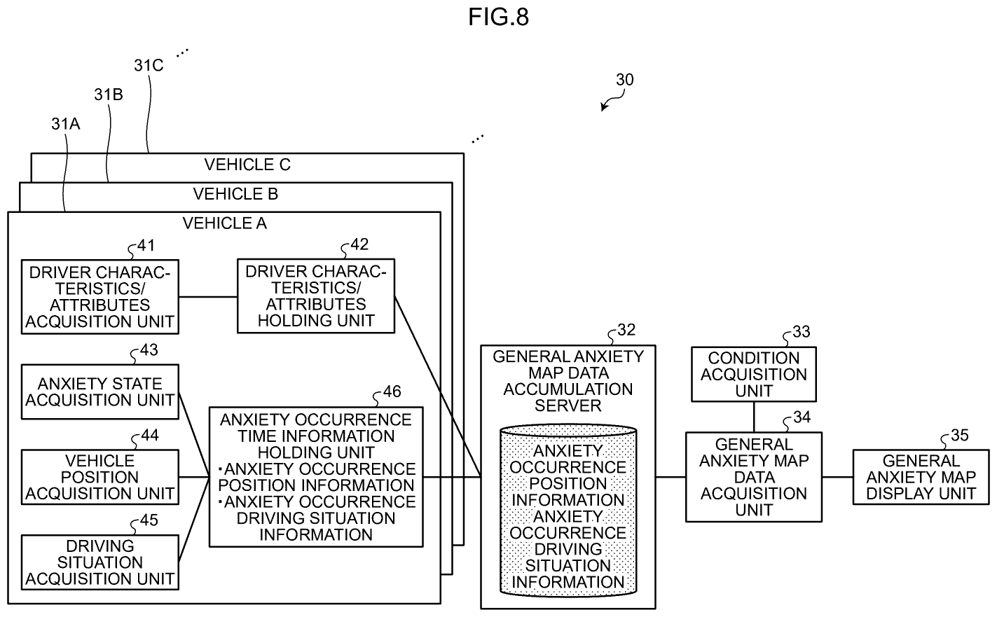

FIG. 8 is a functional configuration block diagram of an anxiety map display system according to the second embodiment.

The anxiety map display system 30 includes information acquisition devices 31A, 31B, 31C, and . . . mounted on a plurality of vehicles A, B, C, and . . . respectively, a general anxiety map data accumulation server 32, a condition acquisition unit 33, a general anxiety map data acquisition unit 34, and a general anxiety map display unit 35.

In the above configuration, the condition acquisition unit 33, the general anxiety map data acquisition unit 34, and the general anxiety map display unit 35 can be mounted on each of the plurality of vehicles A, B, C, and . . . , arranged in a predetermined place, or arranged in a plurality of places where display of the general anxiety map is desired, but are illustrated one by one in FIG. 8 for easy understanding.

Since the information acquisition devices 31A, 31B, 31C, and . . . have the same configuration, the information acquisition device 31A will be described as an example.

The information acquisition device 31A includes a driver characteristics/attributes acquisition unit 41, a driver characteristics/attributes holding unit 42, an anxiety state acquisition unit 43, a vehicle position acquisition unit 44, a driving situation acquisition unit 45, and an anxiety occurrence time information holding unit 46.

In the above configuration, the driver characteristics/attributes acquisition unit 41 acquires in advance, as the driver characteristics, the anxiety characteristics indicating the ease of feeling anxiety at the time of driving, the unfavorable driving scene, the unfavorable driving environment, and the like of the driver of the vehicle on which the information acquisition device 31A is mounted.

In addition, the driver characteristics/attributes acquisition unit 41 acquires in advance, as attributes, a size, an engine displacement, and a type (ordinary automobile, light automobile, truck, and the like) of the vehicle on which the information acquisition device 31A is mounted.

The driver characteristics/attributes holding unit 42 holds the driver characteristics and attributes acquired by the driver characteristics/attributes acquisition unit 41, and uploads (transfers) the held driver characteristics and attributes to the general anxiety map data accumulation server 32 at a predetermined timing or based on a request from the general anxiety map data accumulation server 32.

The anxiety state acquisition unit 43 acquires anxiety state information indicating the anxiety state of the driver of the vehicle on which the anxiety state acquisition unit 43 is mounted.

The anxiety state acquisition unit 43 acquires, for example, biological information such as heart rate of the occupant or electro dermal activity (EDA) of the occupant, and acquires the anxiety state based on the acquired biological information.

More specifically, the anxiety state acquisition unit 43 sequentially acquires, for example, information indicating the heart rate of the occupant and information indicating EDA from an electrode attached to the steering wheel.

Note that the heart rate and EDA of the occupant can also be acquired from a wearable device worn on a wrist, an arm, a head, or the like of the occupant, for example, a smartwatch or the like.

In addition, the algorithm for acquiring the anxiety state is similar to that of the first embodiment.

Note that, as a method for acquiring the anxiety state, in addition to the method for automatically detecting the anxiety state by the biological information (the above example) or the like, the voice uttered by the user, who is the driver, indicating an anxiety state may be recognized, and it may be recognized that the user is in an anxiety state, or a self-reported utterance of anxiety may be acquired by pressing a button.

The general anxiety map data accumulation server 32 acquires the driver characteristics and attributes of all of the information acquisition devices 31A, 31B, 31C, and . . . , which are targets to be managed, and the anxiety occurrence information (the anxiety occurrence position information and the anxiety occurrence driving situation information) acquired from the information acquisition devices 31A, 31B, 31C, and . . . , and accumulates the anxiety occurrence information in association with each other.

The condition acquisition unit 33 receives input from the user regarding the conditions for generating the general anxiety map.

As the condition, for example, a traffic condition (for example, a narrow road, a right turn, a highway, or the like) in which the general user is likely to feel anxiety is specified.

The general anxiety map data acquisition unit 34 makes an inquiry to the general anxiety map data accumulation server 32, acquires, from the general anxiety map data accumulation server 32, anxiety map data for displaying the general anxiety map for a place where the general driver is likely to feel anxiety on the corresponding map screen based on the condition acquired by the condition acquisition unit 33 and the route which is a target to be navigated, and outputs the acquired anxiety map data to the general anxiety map display unit 35.

The general anxiety map display unit 35 is configured as, for example, a display, and displays a general anxiety map considered to be applied to a general driver based on the general anxiety map data acquired by the general anxiety map data acquisition unit 34.

Next, an operation of the second embodiment will be described.

First, operations of the information acquisition devices 31A, 31B, 31C, and . . . will be described.

As described above, since the information acquisition devices 31A, 31B, 31C, and . . . have the same configuration, the operation will be mainly described using the information acquisition device 31A as an example.

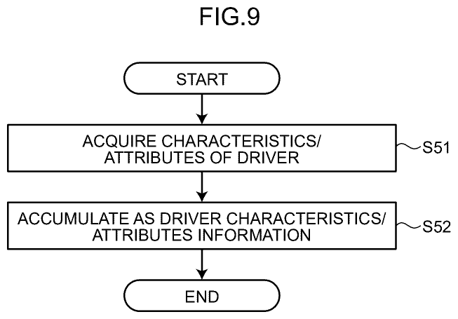

FIG. 9 is an operation processing flowchart at the time of acquiring driver characteristics and attributes.

The driver characteristics/attributes acquisition unit 41 of the information acquisition device 31A displays the driver characteristics/attributes input screen, and acquires the driver characteristics and the attribute from the corresponding driver (step S51).

The driver characteristics include, for example, whether or not the driver is likely to be anxious, a driving situation unfavorable to the driver (driving on narrow roads, right turns, multi-lane driving, merging, high-speed driving, or driving on first-time-driving roads), and a driving environment unfavorable to the driver (weather, nighttime, or the like).

In addition, examples of the attributes include a vehicle type (mini-vehicles, small vehicles, medium and large vehicles, buses, or trucks) and a vehicle configuration (manual vehicle, automatic vehicle, right handle, left handle, or the like).

When acquiring the driver characteristics and attributes, the driver characteristics/attributes acquisition unit 41 of the information acquisition device 31A outputs the acquired driver characteristics and attributes to the driver characteristics/attributes holding unit 42.

As a result, the driver characteristics/attributes holding unit accumulates and holds the input driver characteristics and attributes as driver characteristics/attributes information (step S52).

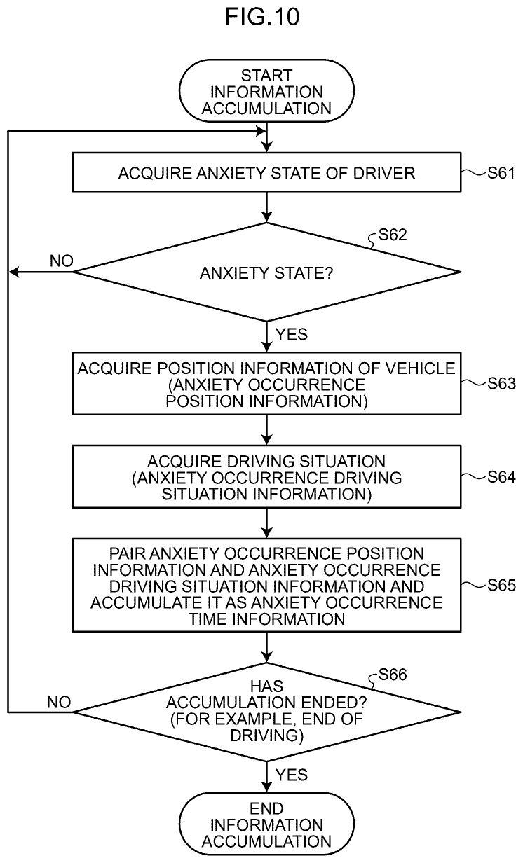

FIG. 10 is an operation processing flowchart at the time of information accumulation.

The anxiety state acquisition units 43 of each of the information acquisition devices 31A, 31B, 31C, and . . . start the operation of acquiring anxiety state information indicating the anxiety state of the driver of the vehicle on which each of the information acquisition devices 31A, 31B, 31C, and . . . is mounted (step S61).

As a result, each anxiety occurrence time information holding unit 46 determines whether or not the anxiety state is acquired in the anxiety state acquisition unit 43 (step S62).

In the determination of step S62, when the anxiety state has not been acquired yet (step S62; No), the process proceeds to step S61 and enters a standby state.

In the determination of step S62, when the anxiety state is acquired (step S62; Yes), the anxiety occurrence time information holding unit 46 acquires the anxiety occurrence position information which is the position information of the own vehicle from the vehicle position acquisition unit 44 (step S63).

Further, the anxiety occurrence time information holding unit 46 acquires the anxiety occurrence driving situation information which is the driving situation of the own vehicle from the driving situation acquisition unit 45 (step S64).

Subsequently, the anxiety occurrence time information holding unit 46 pairs the acquired anxiety occurrence position information and the anxiety occurrence driving situation information and accumulates the information as the anxiety occurrence time information together with the date and time information (step S65).

Then, the anxiety occurrence time information holding unit 46 determines whether or not the accumulation end timing of anxiety occurrence time information has been reached, for example, whether or not the driving has ended (step S66).

In the determination of step S66, when the accumulation end timing of anxiety occurrence time information has not been reached (step S66; No), the information acquisition devices 31A, 31B, 31C, and . . . proceed the processing to step S11 again, and thereafter, the above-described processing is executed.

In the determination of step S66, when the accumulation end timing of the anxiety occurrence time information has been reached (step S66; Yes), the anxiety occurrence time information holding unit 46 ends the information accumulation processing.

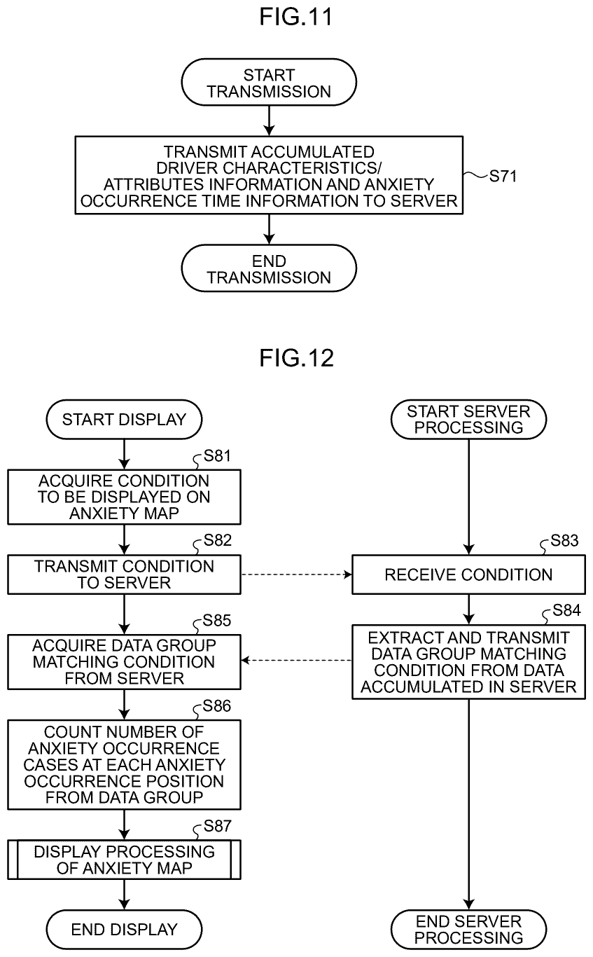

FIG. 11 is a processing flowchart of accumulated information transmission processing.

Subsequently, when the information accumulation processing is ended, the anxiety occurrence time information holding unit 46 transmits the accumulated anxiety occurrence time information and the driver characteristics/attributes information held by the driver characteristics/attributes holding unit 42 to the general anxiety map data accumulation server 32 (step S71).

Next, the operation at the time of displaying the general anxiety map will be described.

FIG. 12 is an explanatory diagram of a display processing sequence of the general anxiety map.

In this case, the user who wishes to display the anxiety map inputs a condition of information desired to be displayed on the general anxiety map via the condition acquisition unit 33 via the condition acquisition unit 33 mounted on the own vehicle or the condition acquisition unit 33 arranged at a predetermined place.

Note that a default display information condition can also be used as the condition of the information displayed on the general anxiety map.

As a result, the condition acquisition unit 33 acquires a condition (user input information or default information) for generating the general anxiety map, and outputs the acquired condition to the general anxiety map data acquisition unit 34 (step S81).

As a result, the general anxiety map data acquisition unit 34 transmits the condition for generating the general anxiety map to the general anxiety map data accumulation server 32 (step S82).

As a result, the general anxiety map data accumulation server 32 receives the condition for generating the general anxiety map (step S83).

Then, the general anxiety map data accumulation server 32 extracts a data group matching the condition from the data accumulated in the general anxiety map data accumulation server 32, and transmits the extracted data group to the general anxiety map data acquisition unit 34 (step S84).

The general anxiety map data acquisition unit 34 receives and acquires a data group matching the condition from the general anxiety map data accumulation server 32 (step S85).

Then, the general anxiety map data acquisition unit 34 counts the number of anxiety occurrence cases at each anxiety occurrence position from the acquired data group (step S86).

Here, the anxiety occurrence position is a position corresponding to a link or a node constituting the map data, and in actual processing, processing is performed as information for identifying the link or the node.

Then, as in the case illustrated in FIG. 5 or 6, the general anxiety map data acquisition unit 34 generates and outputs the general anxiety map display data to the general anxiety map display unit 35 to perform superimposition display on the map by the frequency display method according to the number of anxiety occurrence cases counted at the position of the link or the node corresponding to the anxiety occurrence position.

As a result, the general anxiety map display unit 35 performs processing of displaying the general anxiety map as in the case illustrated in FIGS. 7A and 7B (step S87).

As described above, according to the second embodiment, it is possible to display and use an average anxiety map of a general user reflecting characteristics and attributes of an unspecified large number of users (drivers).

Therefore, the user who uses the general anxiety map can drive with a margin even on a first-time-driving road.

In the above description, the general anxiety map data acquisition unit 34 receives and acquires the data group matching the condition from the general anxiety map data accumulation server 32, and counts the number of anxiety occurrence cases at each anxiety occurrence position from the acquired data group. However, the general anxiety map data accumulation server 32 may be configured to have a similar function.

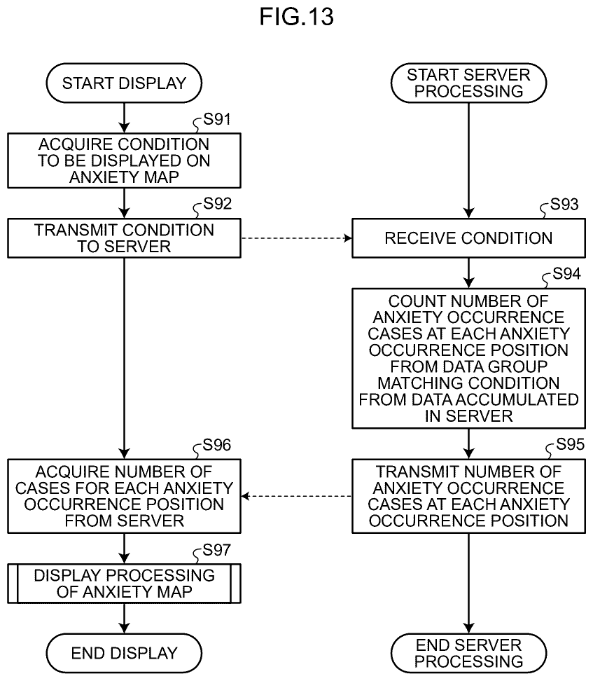

FIG. 13 is an explanatory diagram of a modification example of the display processing sequence of the general anxiety map.

Also in this case, the user who wishes to display the anxiety map inputs a condition of information desired to be displayed on the general anxiety map via the condition acquisition unit 33 via the condition acquisition unit 33 mounted on the own vehicle or the condition acquisition unit 33 arranged at a predetermined place.

The condition acquisition unit 33 acquires a condition (user input information or default information) for generating the general anxiety map, and outputs the acquired condition to the general anxiety map data acquisition unit 34 (step S91).

Then, the general anxiety map data acquisition unit 34 transmits the condition for generating the general anxiety map to the general anxiety map data accumulation server 32 (step S92).

As a result, the general anxiety map data accumulation server 32 receives the condition for generating the general anxiety map (step S93).

Then, the general anxiety map data accumulation server 32 extracts a data group matching the condition from the data accumulated in the general anxiety map data accumulation server 32. Then, the general anxiety map data accumulation server 32 counts the number of anxiety occurrence cases at each anxiety occurrence position from the extracted data group (step S94).

Subsequently, as in the case illustrated in FIG. 5 or 6, the general anxiety map data accumulation server 32 transmits data in which the number of anxiety occurrence cases is associated with each of the anxiety occurrence positions to the general anxiety map data acquisition unit 34 in order to perform superimposition display on the map by the frequency display method according to the number of anxiety occurrence cases counted at the position of the link or the node corresponding to the anxiety occurrence position (step S95).

As in the case illustrated in FIG. 5 or 6, the acquired general anxiety map data acquisition unit 34 receives data in which the number of anxiety occurrence cases is associated with each of the anxiety occurrence positions, and generates and outputs the general anxiety map display data to the general anxiety map display unit 35 to perform superimposition display on the map by the frequency display method according to the number of anxiety occurrence cases counted at the position of the link or the node corresponding to the anxiety occurrence position (step S96).

As a result, the general anxiety map display unit 35 performs processing of displaying the general anxiety map as in the case illustrated in FIGS. 7A and 7B (step S97).

As described above, according to the present modification example as well, it is possible to display and use the average anxiety map of the general user reflecting the characteristics and attributes of an unspecified large number of users (drivers), and the user using the general anxiety map can drive with a margin even on the first-time-driving road.

### (3) Third Embodiment

In each of the above embodiments, it has been described that the acquired anxiety occurrence position information and the corresponding anxiety occurrence driving situation information are all correct information and are all accumulated. However, actually, the obtained anxiety occurrence position information and the corresponding anxiety occurrence driving situation information are obtained in a noise manner and may not be useful.

Therefore, in the present third embodiment, by discriminating the unuseful anxiety occurrence position information and the corresponding anxiety occurrence driving situation information, and excluding the unuseful anxiety occurrence position information and the corresponding anxiety occurrence driving situation information from use, it is possible to display an anxiety map that more accurately reflects actual conditions.

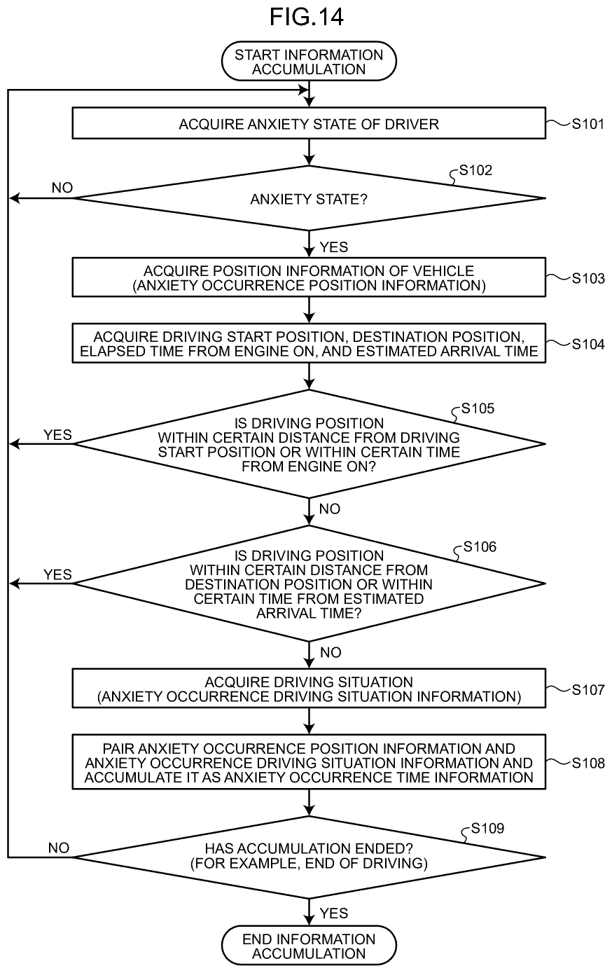

FIG. 14 is a processing flowchart of information accumulation processing of accumulating anxiety occurrence time information according to the third embodiment.

Hereinafter, the description will be given using the anxiety map display device 10 of the first embodiment illustrated in FIG. 1 as an example of the anxiety map display device, but the same applies to the information acquisition devices 31A, 31B, 31C, and . . . .

First, the anxiety state acquisition unit 11 of the anxiety map display device 10 starts an operation of acquiring anxiety state information indicating the anxiety state of the driver of the vehicle (step S101).

As a result, the anxiety occurrence time information holding unit 14 determines whether or not the anxiety state is acquired in the anxiety state acquisition unit 11 (step S102).

In the determination of step S102, when the anxiety state has not been acquired yet (step S102; No), the process proceeds to step S101 and enters a standby state.

In the determination of step S102, when the anxiety state is acquired (step S102; Yes), the anxiety occurrence time information holding unit 14 acquires the anxiety occurrence position information which is the position information of the own vehicle from the vehicle position acquisition unit 12 (step S103).

Next, the anxiety occurrence time information holding unit 14 acquires a driving start position, a driving destination position, an elapsed time from start of engine driving (engine on), and an estimated arrival time of the vehicle on which the own device is mounted (step S104).

In this case, the driving start position, the driving destination position, and the estimated arrival time of the vehicle are acquired by communication from a navigation application of an information processing device such as an in-vehicle navigation system or a smartphone of the user. Similarly, the driving position of the vehicle is also acquired by communication from a navigation application of an information processing device such as an in-vehicle navigation system or a smartphone of the user. The start of engine driving is acquired from an in-vehicle ECU or the like.

Next, the anxiety occurrence time information holding unit 14 determines whether or not the traveling of the vehicle is within a certain distance from the driving start position or within a certain time from the start of engine driving (step S105).

This is because there is a possibility that the degree of tension of the driver is generally high during a predetermined period immediately after the start of driving, it is considered that it is difficult to acquire a stable anxiety state, and there is a tendency for the driver to be more prone to anxiety even in the same driving situation. Therefore, the anxiety occurrence position information and the anxiety occurrence driving situation information acquired under such situations are considered to have low reliability.

In the determination of step S105, when the travel of the vehicle is within a certain distance from the driving start position or within a certain time from the start of engine driving (step S105; Yes), the anxiety occurrence time information holding unit 14 proceeds the processing to step S101 again and repeats the above-described processing not to acquire the anxiety occurrence position information and the anxiety occurrence driving situation information.

In the determination of step S105, when the travel of the vehicle exceeds a certain distance from the driving start position or exceeds a certain time from the start of engine driving (step S105; No), the anxiety occurrence time information holding unit 14 determines whether or not the driving position of the vehicle is within a certain distance from the destination position or within a certain time from the estimated arrival time (step S106).

This is because, during the predetermined period immediately before arrival at the destination, the degree of tension of the driver may increase again unlike the normal traveling state on the intermediate route, and it is considered difficult to acquire a stable anxiety state.

Therefore, the anxiety occurrence position information and the anxiety occurrence driving situation information acquired under such situations are also considered to have low reliability.

In the determination of step S106, when the driving position of the vehicle is within a certain distance from the destination position or within a certain time from the estimated arrival time (step S106; Yes), the anxiety occurrence time information holding unit 14 proceeds the processing to step S101 again and repeats the above-described processing not to acquire the anxiety occurrence position information and the anxiety occurrence driving situation information.

In the determination in step S106, when the driving position of the vehicle exceeds a certain distance from the destination position or exceeds a certain time until the estimated arrival time (step S106; No), the anxiety occurrence time information holding unit 14 acquires the anxiety occurrence driving situation information which is the driving situation of the own vehicle from the driving situation acquisition unit 13 (step S107).

Subsequently, the anxiety occurrence time information holding unit 14 pairs the acquired anxiety occurrence position information and the anxiety occurrence driving situation information and accumulates the information as the anxiety occurrence time information together with the date and time information (step S108).

Then, the anxiety occurrence time information holding unit 14 determines whether or not the accumulation end timing of anxiety occurrence time information has been reached, for example, whether or not the driving has ended (step S109).

In the determination of step S109, when the accumulation end timing of anxiety occurrence time information has not been reached (step S109; No), the processing proceeds to step S101 again, and thereafter, the above-described processing is executed.

In the determination of step S109, when the accumulation end timing of the anxiety occurrence time information has been reached (step S109; Yes), the anxiety occurrence time information holding unit 14 ends the processing.

Since the details of the anxiety occurrence information have not been described in each of the above embodiments, the anxiety occurrence time information data storing the anxiety occurrence time information will be described here.

In the following example, the anxiety occurrence time information data in a case where an anxiety state is detected in a specific node will be described.

Hereinafter, the description will be given using the anxiety map display device 10 of the first embodiment illustrated in FIG. 1 as an example of the anxiety map display device, but the same applies to the information acquisition devices 31A, 31B, 31C, and . . . .

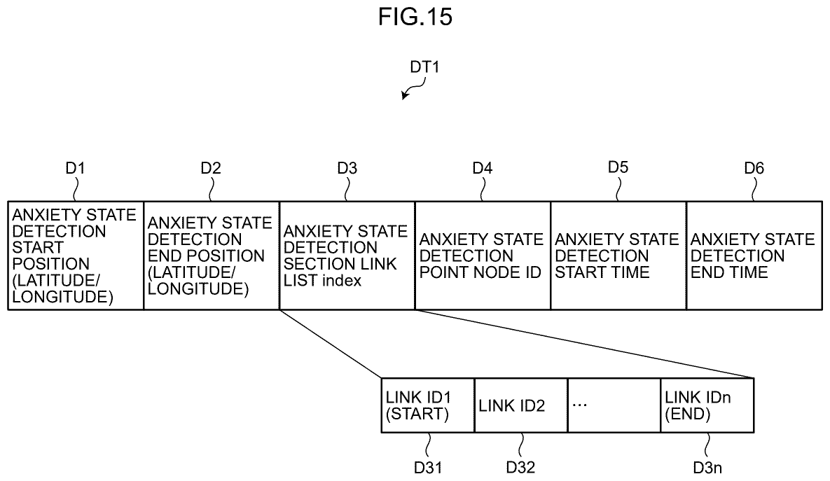

FIG. 15 is an explanatory diagram of an example of the anxiety occurrence time information data.

Anxiety occurrence time information data DT1 includes anxiety state detection start position data D1, anxiety state detection end position data D2, anxiety state detection section link list index data D3, anxiety state detection point node ID data D4, anxiety state detection start time data D5, and anxiety state detection end time data D6.

The anxiety state detection start position data D1 stores, as latitude data and longitude data, a position where detection of a series of anxiety states is started in the anxiety state acquisition unit 11.

The anxiety state detection end position data D2 stores, as latitude data and longitude data, a position where detection of a series of anxiety states is ended in the anxiety state acquisition unit 11.

The anxiety state detection section link list index data D3 stores, as link ID data D31 to D3n, links ID1 to IDn which are data for identifying links of map data corresponding to an anxiety state detection start position corresponding to the anxiety occurrence time information data DT1 and an anxiety state detection end position corresponding to the anxiety state detection end position data D2.

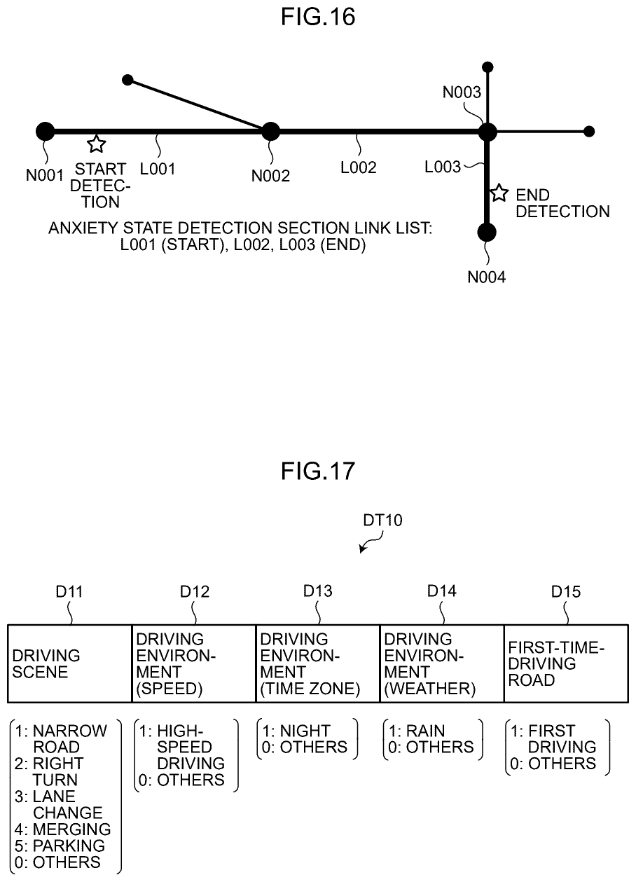

FIG. 16 is an explanatory diagram of an anxiety state section link list.

In the example of FIG. 16, the node N001 is a driving start point, and the node N004 is a destination point.

Therefore, in the case of the example of FIG. 16, the link ID data D31 to D33 for identifying the link L001, the link L002, and the link L003 are stored in the anxiety state detection section link list index data D3.

The actual detection start of the anxiety occurrence state is at the position marked with an asterisk corresponding to the link L001, where the driver's state is considered to have transitioned to a normal state, making reliable detection of the anxiety state possible. The actual detection end of the anxiety occurrence state is at the position marked with an asterisk corresponding to the link L004, where the driver's state is considered to have transitioned back to a tense state, making reliable detection of the anxiety state difficult.

The anxiety state detection point node ID data D4 stores data for identifying a node corresponding to the anxiety state detection point where the anxiety data is actually detected.

For example, in a case where the anxiety state detection point where the anxiety data is actually detected is the node N003 corresponding to the intersection turning right, data for identifying the node N003 is stored in the anxiety state detection point node ID data D4.

Note that, in the present example, the anxiety state is detected at a point corresponding to the node. However, in a case where the anxiety state is detected at a point corresponding to a link such as driving on a narrow road or highway driving, an index to the anxiety state detection point link ID data or the link ID list may be stored instead of the anxiety state detection point node ID data D4.

The anxiety state detection start time data D5 stores data for identifying the time at which the detection of the anxiety state has started.

The anxiety state detection end time data D6 stores data for identifying the time when the detection of the anxiety state continuously detected after the detection of the anxiety state is started is ended.

Therefore, the anxiety map can be easily displayed by collecting the anxiety occurrence time information data DT1.

Next, conditions input by the user to display the anxiety map will be described in detail.

FIG. 17 is an explanatory diagram of a data format of condition data output by a condition data acquisition unit.

The condition input in the condition acquisition unit 15 is output to the anxiety map data generation unit 16 as condition data DT10. The condition input in the condition acquisition unit 33 is output to the general anxiety map data acquisition unit 34.

In this case, the condition data DT10 includes, as an example, driving scene data D11, driving environment (speed) data D12, driving environment (time zone) data D13, driving environment (weather) data D14, and first-time-driving road data D15.

As the driving scene data D11, a driving scene to be displayed on the anxiety map is set. For example, narrow road=1, right turn=2, lane change=3, merging=4, parking=5, and others=0 are set as data values.

As the driving environment (speed) data D12, information related to the speed of the vehicle is set as the driving environment desired to be displayed on the anxiety map. For example, highway (road) driving=1 and others (ordinary roads)=1 are set as data values.

As the driving environment (time zone) data D13, information regarding the time zone is set as the driving environment desired to be displayed on the anxiety map. For example, nighttime=1 and others=0 are set as data values.

As the driving environment (weather) data D14, information regarding the weather is set as the driving environment desired to be displayed on the anxiety map. For example, rain=1 and others=0 are set as data values.

As the first-time-driving road data D15, information on whether or not the road is a first-time-driving road is set. For example, first driving (driving road)=1 and others=0 are set as data values.

Next, an example of a condition acquisition screen in the condition data acquisition unit will be described.

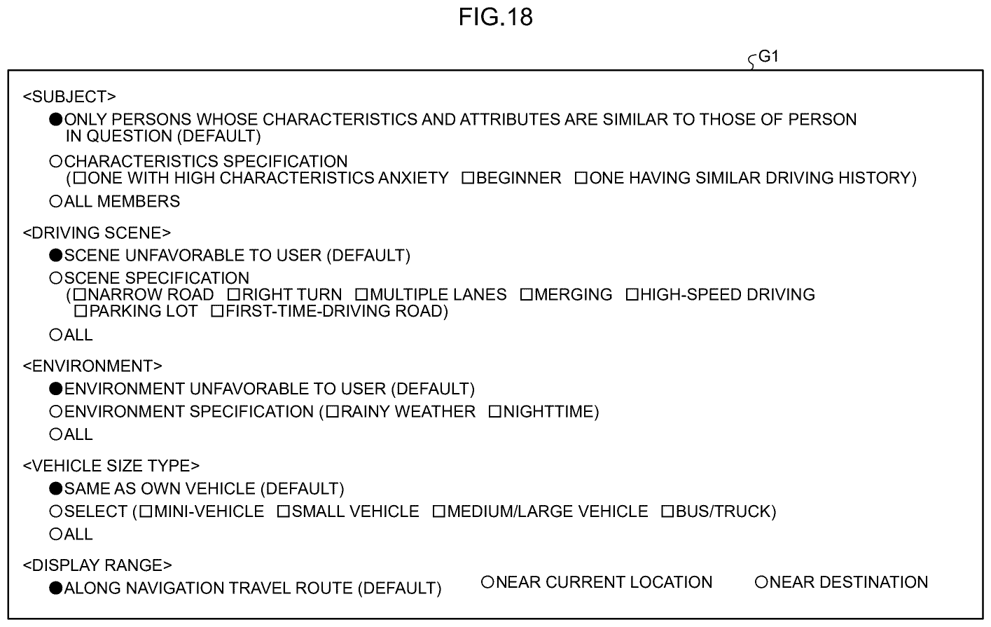

FIG. 18 is an explanatory diagram of an example of a condition acquisition screen in a case where the general anxiety map is displayed.

A condition acquisition screen G1 is for displaying the general anxiety map.

In the condition acquisition screen G1, the subject, the driving scene, the (driving) environment, the size type of the vehicle, and the display range are specified in order to identify data necessary for displaying the general anxiety map.

In the example of FIG. 18, only a person having characteristics and attributes similar to the characteristics and attributes of the user himself or herself is set as the subject as the default (value).

In addition, it is possible to specify characteristics. In the example of FIG. 18, a person with high anxiety characteristics, a beginner, and a person with similar driving history can be selected.

In addition, all the persons can be set as the subject.

Furthermore, in the case of the example of FIG. 18, as the driving scene, a driving scene unfavorable to the user himself or herself is set as a default (value).

Further, it is possible to specify a driving scene. In the example of FIG. 18, it is possible to select a narrow road, a right turn, a negative lane, merging, high-speed driving, a parking lot, or a first-time-driving road.

In addition, all driving scenes can be set.

In the case of the example of FIG. 18, as the vehicle size type, the same vehicle size type as the user's own vehicle is set as a default (value).

In addition, it is possible to specify the size type of the vehicle. In the example of FIG. 18, a mini-vehicle, a small vehicle, a medium/large vehicle, and a bus/truck can be selected.

In addition, it is possible to set the size types of all the vehicles.

In each of the above embodiments, the details of the anxiety occurrence position information and the anxiety occurrence driving situation information have not been described, and thus, are held in the anxiety occurrence time information holding unit 14.

Here, the setting of the characteristics and attributes of the person in question will be described.

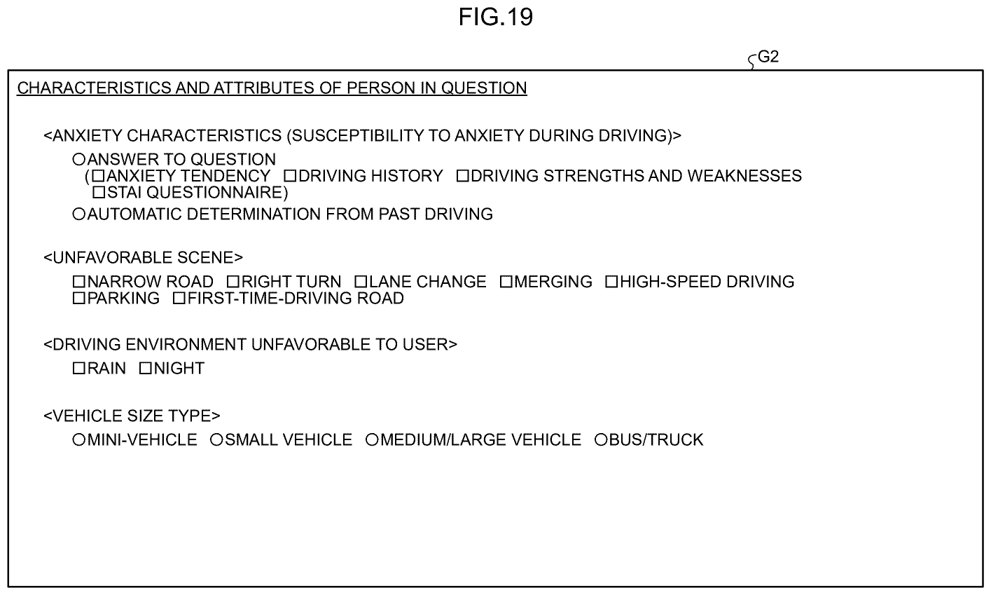

FIG. 19 is an explanatory diagram of a setting screen for characteristics and attributes of the person in question.

In the setting screen G2 of the characteristics and attributes of the person in question, anxiety characteristics (ease of anxiety at the time of driving), the unfavorable scene (unfavorable driving scene), the unfavorable driving environment, and a size type of the vehicle are specified.

In the case of FIG. 19, either an answer to a question or automatic determination from past driving can be selected as the anxiety characteristics (ease of anxiety at the time of driving).

In a case where an answer is selected for the question, it is possible to select one or more of an anxiety tendency, a driving history, and driving strengths and weaknesses, or from the STAI questionnaire, as a reason for the person's anxiety. Here, the STAI questionnaire is a questionnaire of the anxiety measurement test, and selecting this means that the selection is based on the result of the anxiety measurement test.

In a case where the automatic determination is selected from the past driving, the frequency or the like at which the anxiety state is detected in the past driving of the user is compared with a predetermined reference value to estimate the anxiety characteristics.

In the case of the example of FIG. 19, one or a plurality of scenes can be selected from a narrow road, a right turn, a lane change, merging, highway (road) driving, parking, and a first-time-driving road as unfavorable scenes.

In the case of the example of FIG. 19, rainy weather or nighttime can be selected as the unfavorable driving environment.

In the case of FIG. 19, as the size type of the vehicle, any one of a mini-vehicle, a small vehicle, a medium/large vehicle, and a bus/truck can be selected.

Next, a method for deriving the anxiety characteristics of the user will be described.

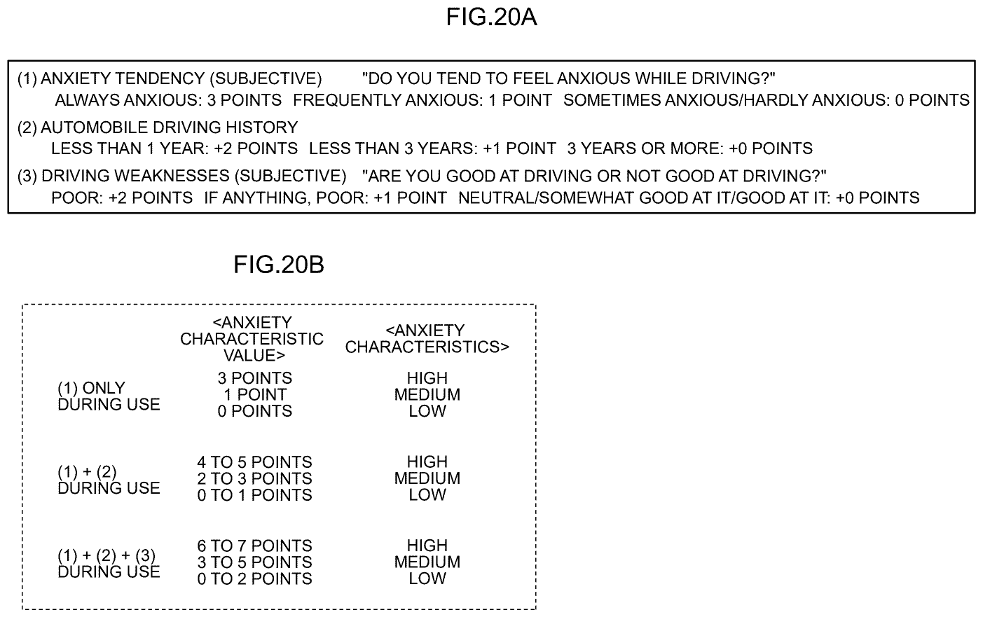

FIGS. 20A and 20B are explanatory diagrams of a derivation method in a case of deriving the anxiety characteristics of a user from the user's responses to questions.

In FIGS. 20A and 20B, for easy understanding, minimum questions are described, but the present invention is not limited thereto.

As illustrated in FIG. 20A, question contents include the following three categories:
(1) anxiety tendency (subjective),
(2) automobile driving history, and
(3) driving weaknesses (subjective).

However, the (1) anxiety tendency (subjective) is considered as a minimum required question.

More specifically, for the (1) anxiety tendency (subjective), for example, an answer of always anxious, frequently anxious, and sometimes anxious/hardly anxious can be selected for the question of “Do you tend to feel anxious while driving?”, and the following anxiety characteristic values are obtained. In the following description, it is assumed that the higher the value of the anxiety characteristic value, the higher the anxiety characteristics.
Always anxious: 3 points
Frequently anxious: 1 point
Sometimes anxious/hardly anxious: 0 points

In addition, for the (2) automobile driving history, the following answers can be selected, and the following anxiety characteristic value (addition amount) is obtained.
Less than 1 year: +2 points
Less than 3 years: +1 point
3 years or more: +0 points

In addition, for the (3) driving weaknesses (subjective), the following answers can be selected, and the following anxiety characteristic value (addition amount) is obtained.
Not good at it: +2 points
Somewhat not good at it: +1 point
Neutral/Somewhat good at it/Good at it: +0 points

FIG. 20B is a correspondence explanatory diagram of the anxiety characteristic value and the determination result of the anxiety characteristics.

As illustrated in FIG. 20B, in a case where only the (1) anxiety tendency (subjective) is used for determination, it is determined that the anxiety characteristics are high when the anxiety characteristic value is 3 points, it is determined that the anxiety characteristics are moderate when the anxiety characteristic value is 1 point, and it is determined that the anxiety characteristics are low when the anxiety characteristic value is 0 points.

In addition, as illustrated in FIG. 20B, in a case where the (1) anxiety tendency (subjective) and the (2) automobile driving history are used for determination, it is determined that the anxiety characteristics are high when the anxiety characteristic value is 4 to 5 points, it is determined that the anxiety characteristics are moderate when the anxiety characteristic value is 2 to 3 points, and it is determined that the anxiety characteristics are low when the anxiety characteristic value is 0 to 1 points.

In addition, as illustrated in FIG. 20B, in a case where the (1) anxiety tendency (subjective), the (2) automobile driving history, and (3) driving weaknesses (subjective) are used for determination, it is determined that the anxiety characteristics are high when the anxiety characteristic value is 6 to 7 points, it is determined that the anxiety characteristics are moderate when the anxiety characteristic value is 3 to 5 points, and it is determined that the anxiety characteristics are low when the anxiety characteristic value is 0 to 2 points.

As described above, according to the third embodiment, it is possible to collect the anxiety occurrence position information and the anxiety occurrence driving situation information with higher reliability, and eventually, it is possible to display the anxiety display map with higher reliability.

### (4) Fourth Embodiment

Each of the above embodiments relates to an anxiety map display device or an anxiety map display system, but the present fourth embodiment relates to anxiety feature quantities presentation device that more accurately presents anxiety feature quantities to a user.

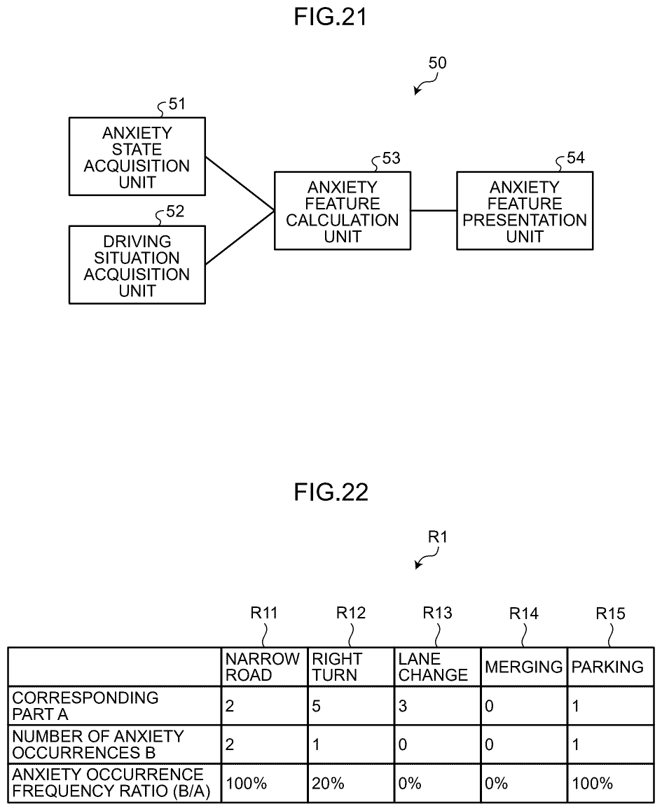

FIG. 21 is a functional configuration block diagram of the anxiety feature quantities presentation device.

An anxiety feature quantities presentation device 50 includes an anxiety state acquisition unit 51, a driving situation acquisition unit 52, an anxiety feature calculation unit 53, and an anxiety feature presentation unit 54.

The anxiety state acquisition unit 51 acquires anxiety state information indicating an anxiety state of a driver of a vehicle.

The anxiety state acquisition unit 51 acquires, for example, biological information such as heart rate of the occupant or electro dermal activity (EDA) of the occupant, and acquires the anxiety state based on the acquired biological information.

The driving situation acquisition unit 52 acquires the driving situation of the own vehicle based on the route search result, the map matching result, and the like. Here, similarly to the first embodiment, the driving situation is a driving situation that may cause an anxiety state such as a width, a lane, a type of a road (an automobile-only road including a highway or a general road) on which the own vehicle is traveling, a right turn, a lane change, merging, parking, traffic congestion information provided by a traffic control system, nighttime driving acquired from time information, and rainy weather driving detected by wiper operation or sensors. However, the present invention is not limited thereto.

The anxiety feature calculation unit 53 calculates anxiety feature quantities for each predetermined aggregation unit.

Here, the predetermined aggregation unit is a driving scene, a driving environment, a driving scene in which anxiety is generally less likely to occur, the presence or absence of passengers, a first-time-driving road, a predetermined period immediately after the start of driving, and the like, and the number of anxiety occurrences is counted for each aggregation unit to calculate an anxiety occurrence ratio for each aggregation unit.

The anxiety feature presentation unit 54 presents the anxiety feature obtained based on the calculation result of the anxiety feature calculation unit 53 to the user.

FIG. 22 is a diagram for describing an example of calculation results of the anxiety feature quantities for each driving scene.

As illustrated in FIG. 22, in the anxiety feature quantities calculation result R1, an anxiety occurrence frequency ratio (B/A %) is calculated based on (the number of) parts A corresponding to a predetermined driving scene (a narrow road, a right turn, a lane change, merging, and parking) which is a predetermined aggregation unit and the number of anxiety occurrences B of the driver at the corresponding portions.

In the example of FIG. 22, anxiety feature quantities calculation result R1 indicates anxiety feature quantities calculation result R11 for a narrow road, anxiety feature quantities calculation result R12 for a right turn, anxiety feature quantities calculation result R13 for a lane change, anxiety feature quantities calculation result R14 for merging, and anxiety feature quantities calculation result R15 for parking.

For example, regarding the anxiety feature quantities calculation result R12 for right turn, there are 5 corresponding parts and the number of times the anxiety state of the driver is detected, that is, the number of anxiety occurrences is 1, which indicates that the anxiety occurrence frequency ratio is 20%.

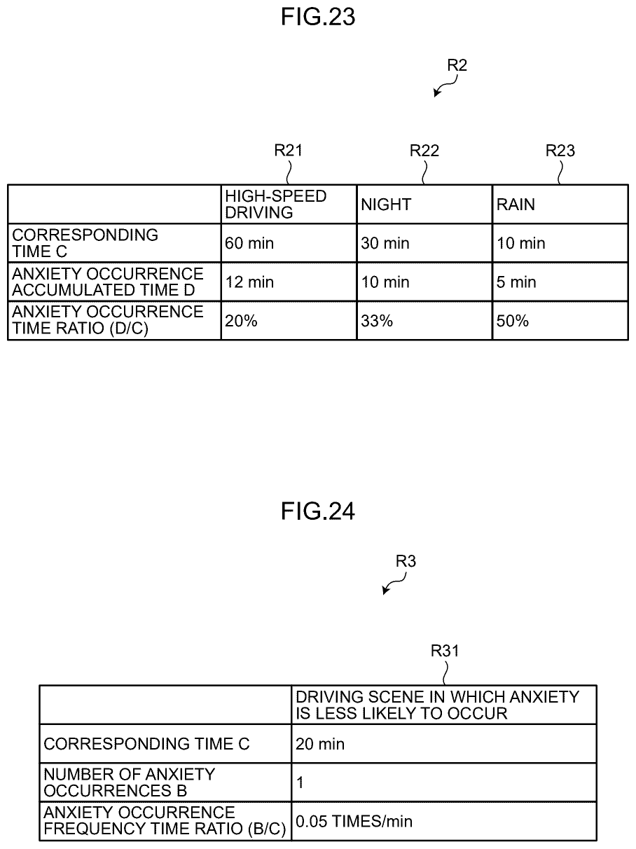

FIG. 23 is a diagram for describing an example of calculation results of the anxiety feature quantities for each driving environment.

As illustrated in FIG. 23, the anxiety feature quantities calculation result R2 calculates the anxiety occurrence time ratio (D/C %) based on the corresponding time C corresponding to a predetermined driving environment (highway (road) driving, nighttime driving, and rainy weather driving) which is a predetermined aggregation unit and the anxiety occurrence accumulated time D of the driver under the corresponding condition.

In the example of FIG. 23, the anxiety feature quantities calculation result R2 indicates the anxiety feature quantities calculation result R21 for highway (road) driving, the anxiety feature quantities calculation result R22 for nighttime driving, and the anxiety feature quantities calculation result R23 for rainy weather driving.

For example, regarding the anxiety feature quantities calculation result R22 for nighttime driving, the corresponding driving time is 30 minutes, and the time when the anxiety state of the driver has been detected, that is, the anxiety occurrence accumulated time is 10 minutes, which indicates that the anxiety occurrence time ratio is 33%.

FIG. 24 is a diagram for describing an example of calculation results of anxiety feature quantities for each driving scene in which anxiety is generally less likely to occur.

As illustrated in FIG. 24, the anxiety feature quantities calculation result R3 calculates the anxiety occurrence frequency time ratio (B/C times/min) based on the corresponding time C corresponding to the driving scene in which the anxiety is generally less likely to occur which is a predetermined aggregation unit and the number of anxiety occurrences B of the driver under the corresponding condition.

In the example of FIG. 24, the anxiety feature quantities calculation result R3 shows anxiety feature quantities calculation result R31 for a driving scene in which anxiety is generally less likely to occur.

Regarding the anxiety feature quantities calculation result R31, the corresponding driving time of the driving scene in which the anxiety is generally less likely to occur is 20 minutes, and the number of times the anxiety state of the driver is detected, that is, the number of anxiety occurrences is 1, which indicates that the anxiety occurrence frequency time ratio is 0.05 times/min.

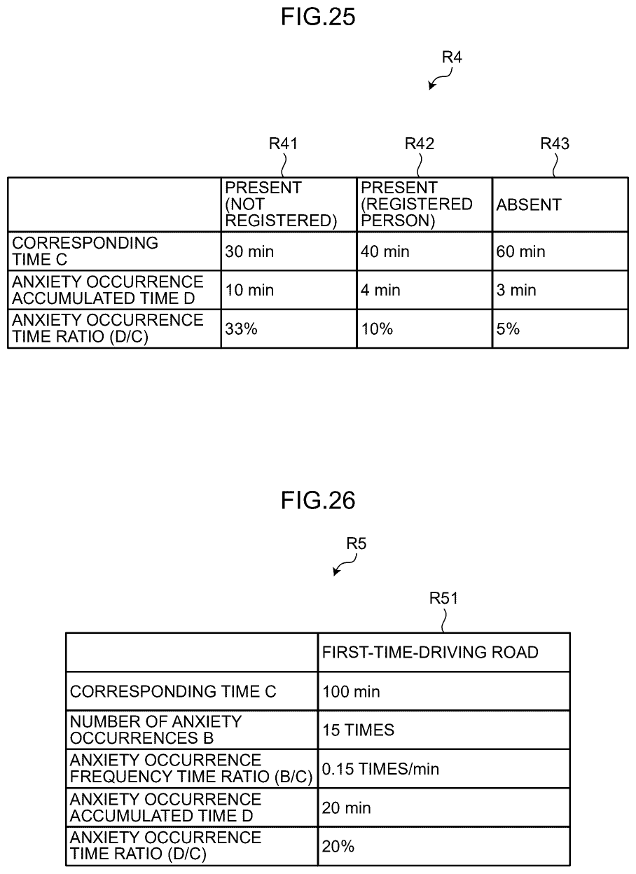

FIG. 25 is a diagram for describing an example of calculation results of anxiety feature quantities for each case of presence or absence of a passenger.

As illustrated in FIG. 25, the anxiety feature quantities calculation result R4 calculates the anxiety occurrence time ratio (D/C %) based on the corresponding time C corresponding to the presence or absence of a passenger (unregistered passenger present, registered passenger present, no passenger) as a predetermined aggregation unit, and the anxiety occurrence accumulated time D of the driver under the corresponding condition.

In the above configuration, the reason for distinguishing between the unregistered passenger and the registered passenger is that the registered passenger is a family member, a friend, an acquaintance, or the like, and even when the registered passenger is a passenger, it is considered that the tension of the driver is small and the occurrence of anxiety is small. On the other hand, the possibility that the unregistered passenger is a temporary passenger who usually does not use the vehicle is high, and it is considered that the tension of the driver may increase and the occurrence of anxiety also increases.

In the example of FIG. 25, the anxiety feature quantities calculation result R4 indicates anxiety feature quantities calculation result R41 in a case where there is an unregistered passenger, anxiety feature quantities calculation result R42 in a case where there is a registered passenger, and anxiety feature quantities calculation result R43 in a case where there is no passenger.

For example, regarding the anxiety feature quantities calculation result R42 for a case where there is a registered passenger, the corresponding driving time is 40 minutes, and the time when the anxiety state of the driver has been detected, that is, the anxiety occurrence accumulated time is 4 minutes, which indicates that the anxiety occurrence time ratio is 10%.

FIG. 26 is a diagram for describing an example of calculation results of anxiety feature quantities for each first-time-driving road.

As illustrated in FIG. 26, the anxiety feature quantities calculation result R5 calculates the corresponding time C corresponding to the driving time on the first-time-driving road, the number of anxiety occurrences B, the anxiety occurrence frequency time ratio, the anxiety occurrence accumulated time D, and the anxiety occurrence time ratio (D/C %).

In the example of FIG. 26, the anxiety feature quantities calculation result R5 indicates anxiety feature quantities calculation result R51 in the case of the first-time-driving road.

Regarding the anxiety feature quantities calculation result R51, the corresponding driving time C is 100 minutes, and the number of times B that the anxiety state of the driver was detected is 15, which indicates the anxiety occurrence frequency time ratio (B/C) is 0.15 times/min, and the time during which the anxiety state of the driver was detected, that is, the anxiety occurrence accumulated time D is 20 minutes, which indicates anxiety occurrence time ratio (D/C) is 20%.

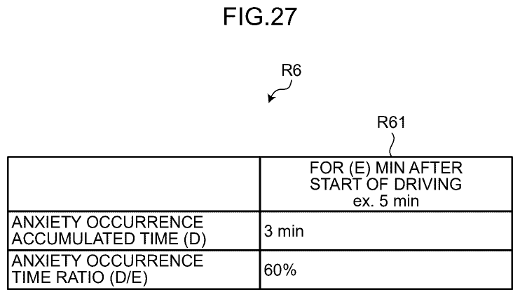

FIG. 27 is a diagram illustrating an example of calculation results of anxiety feature quantities for each predetermined period immediately after the start of driving.

As illustrated in FIG. 27, an anxiety feature quantities calculation result R6 calculates an anxiety occurrence time ratio (D/E %) in a case of, for example, a predetermined period E (for example, 5 minutes) immediately after the start of driving.

In the example of FIG. 27, the anxiety feature quantities calculation result R6 indicates the anxiety feature quantities calculation result R51 in a predetermined period E immediately after the start of driving.

Regarding the anxiety feature quantities calculation result R61, in a case where the predetermined period E immediately after the start of driving is 5 minutes, the time when the anxiety state of the corresponding driver is detected, that is, the anxiety occurrence accumulated time D is 3 minutes, which indicates that the anxiety occurrence time ratio (D/E) is 60%.

Next, an operation of the fourth embodiment will be described.

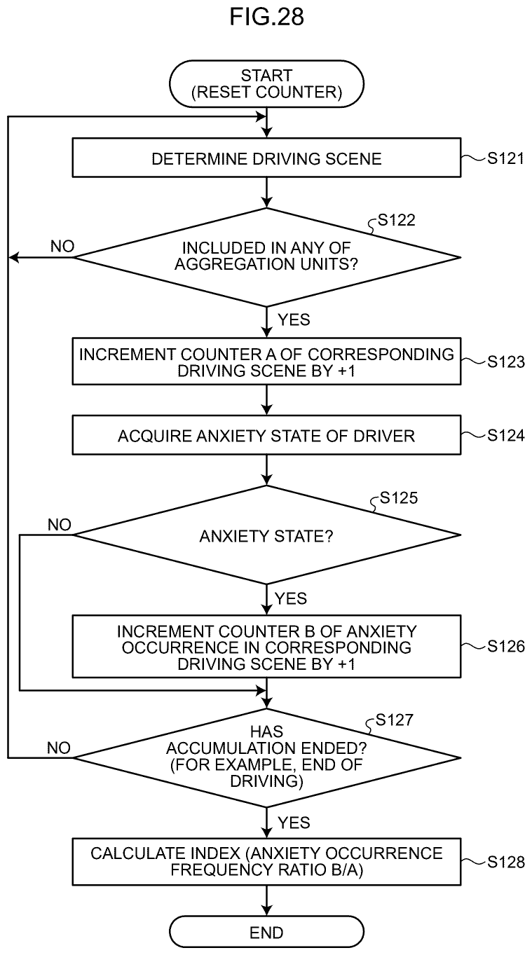

FIG. 28 is a processing flowchart of the processing of calculating the anxiety feature quantities for each driving scene.

In this case, it is assumed that corresponding counters (a driving scene detection number counter A and an anxiety occurrence number counter B) are respectively provided in each driving scene (in the following description, there are five scenes: a narrow road, a right turn, a lane change, merging, and parking).

Prior to the processing of calculating the anxiety feature quantities for each driving scene, the anxiety feature calculation unit 53 resets the counters (the driving scene detection number counter A and the anxiety occurrence number counter B) corresponding to each of all the driving scenes.

First, the anxiety feature calculation unit 53 determines the current driving scene based on the driving situation acquired by the driving situation acquisition unit 52 (step S121).

Subsequently, the anxiety feature calculation unit 53 determines whether or not the determined driving scene is included in any of a narrow road, a right turn, a lane change, merging, and parking (step S122).

In the determination of step S122, when the current driving scene is not included in any of a narrow road, a right turn, a lane change, merging, and parking (step S122; No), the processing proceeds to step S121 again and enters a standby state.

In the determination of step S122, when the current driving scene is included in any of a narrow road, a right turn, a lane change, merging, and parking (step S122; Yes), the driving scene detection number counter A corresponding to the corresponding driving scene is incremented by 1 (+1) (step S123).

Next, the anxiety state acquisition unit 51 acquires the anxiety state of the driver (step S124).

As a result, the anxiety feature calculation unit 53 determines whether or not the driver is in the anxiety state based on the acquisition result of the anxiety state acquisition unit 51 (step S125).

When the driver is not in an anxiety state in step S125 (step S125; No), the processing proceeds to step S127.

When the driver is in an anxiety state in step S125 (step S125; Yes), the anxiety occurrence number counter B corresponding to the corresponding driving scene is incremented by 1 (+1) (step S126).

Next, the anxiety feature calculation unit 53 determines whether or not the accumulation end timing of the anxiety feature quantities for each driving scene has been reached, for example, whether or not the driving has ended (step S127).

In the determination of step S127, when the accumulation end timing of anxiety occurrence time information has not been reached (step S127; No), the anxiety feature calculation unit 53 proceeds the processing to step S121 again, and thereafter, the above-described processing is executed.

In the determination of step S127, when the accumulation end timing of the anxiety feature quantities for each driving scene has been reached (step S127; Yes), the anxiety feature calculation unit 53 calculates an anxiety occurrence frequency ratio (=the number of anxiety occurrences/the number of times driving scene is detected) as an index (step S128), and ends the processing.

As described above, the anxiety feature quantities for each driving scene can be calculated, and appropriate driving or appropriate advice as described later can be presented based on the calculated anxiety feature quantities.

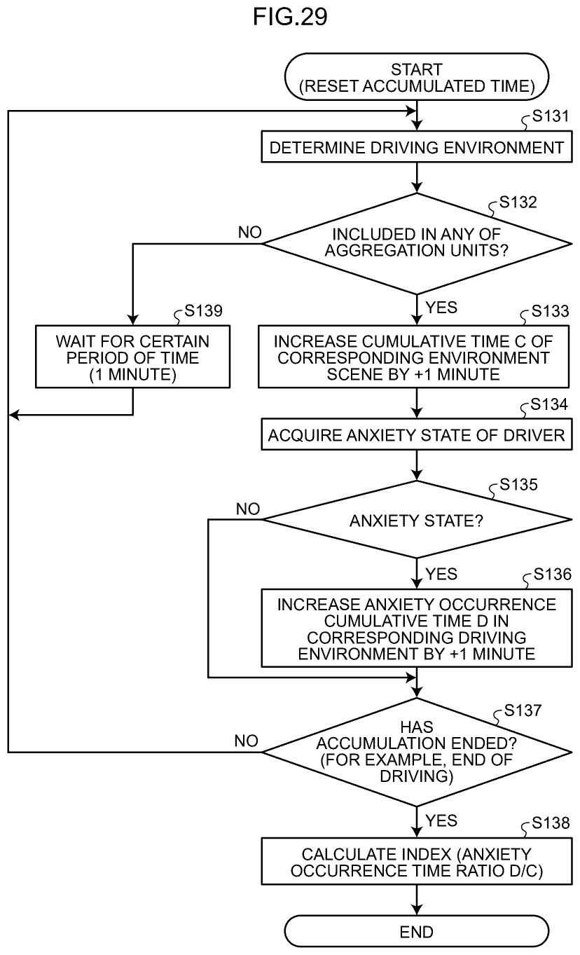

FIG. 29 is a processing flowchart of processing of calculating anxiety feature quantities for each driving environment.

In this case, each driving environment (in the following description, the three environments of highway (road) driving, nighttime driving, and rainy weather driving) is provided with corresponding counters (a driving environment cumulative time counter and an anxiety occurrence cumulative time counter).

Prior to the processing of calculating the anxiety feature quantities for each driving scene, the anxiety feature calculation unit 53 resets the counters (the driving environment cumulative time counter and the anxiety occurrence cumulative time counter) corresponding to each of all the driving scenes.

First, the anxiety feature calculation unit 53 determines the current driving environment based on the driving situation acquired by the driving situation acquisition unit 52 (step S131).

Subsequently, the anxiety feature calculation unit 53 determines whether or not the determined driving environment is included in any of highway (road) driving, nighttime driving, and rainy weather driving (step S132).

In the determination of step S132, when the current driving environment is not included in any of highway (road) driving, nighttime driving, and rainy weather driving (step S132; No), the processing waits for a certain period of time (for example, 1 minute), and processing proceeds to step S131 again.

In the determination of step S132, when the current driving environment is included in any of highway (road) driving, nighttime driving, and rainy weather driving (step S132; Yes), a driving environment cumulative time counter C corresponding to the corresponding driving environment is incremented by 1 (step S133).

Next, the anxiety state acquisition unit 51 acquires the anxiety state of the driver (step S134).

As a result, the anxiety feature calculation unit 53 determines whether or not the driver is in the anxiety state based on the acquisition result of the anxiety state acquisition unit 51 (step S135).

When the driver is not in an anxiety state in step S125 (step S135; No), the processing proceeds to step S137.

When the driver is in an anxiety state in step S135 (step S135; Yes), an anxiety occurrence cumulative time counter D corresponding to the corresponding driving scene is incremented by 1 (step S136).

Next, the anxiety feature calculation unit 53 determines whether or not the accumulation end timing of the anxiety feature quantities for each driving environment has been reached, for example, whether or not the driving has ended (step S137).

In the determination of step S137, when the accumulation end timing of anxiety occurrence time information has not been reached (step S137; No), the anxiety feature calculation unit 53 proceeds the processing to step S131 again, and thereafter, the above-described processing is executed.

In the determination of step S137, when the accumulation end timing of the anxiety feature quantities for each driving scene has been reached (step S137; Yes), the anxiety feature calculation unit 53 calculates an anxiety occurrence time ratio (=the number of anxiety occurrences/the anxiety occurrence cumulative time) as an index (step S138), and ends the processing.

As described above, the anxiety feature quantities for each driving environment can be calculated, and appropriate driving or appropriate advice as described later can be presented based on the calculated anxiety feature quantities.

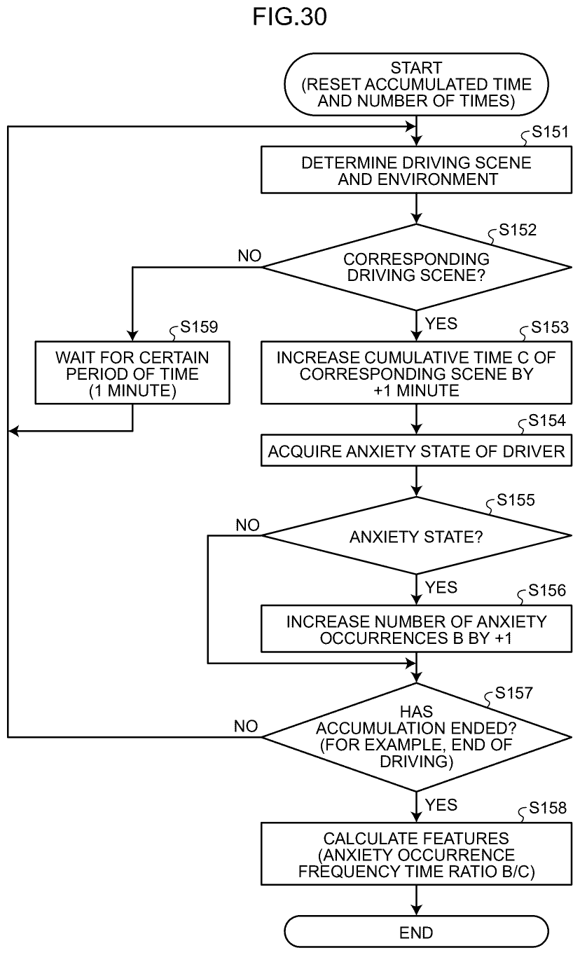

FIG. 30 is a processing flowchart of processing of calculating anxiety feature quantities for each driving scene where anxiety is generally less likely to occur.

In this case, it is assumed that corresponding counters (the driving scene cumulative time counter C and the anxiety occurrence number counter B) are respectively provided for each driving scene (for example, a situation where there is one lane in the same direction, the driving speed is 40 to 60 km, and it does not rain during daytime driving) in which anxiety is generally less likely to occur.

The anxiety feature calculation unit 53 resets the counters (the driving scene cumulative time counter C and the anxiety occurrence number counter B) corresponding to each of all the driving scenes in which anxiety is generally less likely to occur prior to the processing of calculating the anxiety feature quantities for each driving scene in which anxiety is generally less likely to occur.

First, the anxiety feature calculation unit 53 determines the current driving scene and the driving environment based on the driving situation acquired by the driving situation acquisition unit 52 (step S151).

Subsequently, the anxiety feature calculation unit 53 determines whether or not the determined driving scene and the driving environment are included in a driving scene in which anxiety is generally less likely to occur (step S152).

In the determination of step S152, when the current driving scene and the driving environment are not included in the driving scene in which the anxiety is generally less likely to occur (step S152; No), the processing waits for a certain period of time (for example, 1 minute) (step S159), and processing proceeds to step S151 again.

In the determination of step S152, when the current driving scene and the driving environment are included in the driving scene in which the anxiety is generally less likely to occur (step S152; Yes), a driving scene cumulative time counter C corresponding to the corresponding driving scene is incremented by 1 (step S153).

Next, the anxiety state acquisition unit 51 acquires the anxiety state of the driver (step S154).

As a result, the anxiety feature calculation unit 53 determines whether or not the driver is in an anxiety state based on the acquisition result of the anxiety state acquisition unit 51 (step S155).

When the driver is not in an anxiety state in step S125 (step S125; No), the processing proceeds to step S127.

When the driver is in an anxiety state in step S125 (step S125; Yes), the anxiety occurrence number counter B corresponding to the corresponding driving scene is incremented by 1 (+1) (step S156).

Next, the anxiety feature calculation unit 53 determines whether or not the accumulation end timing of the anxiety feature quantities has been reached, for example, whether or not the driving has ended (step S157).

In the determination of step S157, when the accumulation end timing of anxiety feature quantities has not been reached (step S157; No), the anxiety feature calculation unit 53 proceeds the processing to step S151 again, and thereafter, the above-described processing is executed.

In the determination of step S157, when the accumulation end timing of the anxiety feature quantities has been reached (step S157; Yes), the anxiety feature calculation unit 53 calculates an anxiety occurrence frequency time ratio (=the driving scene cumulative time/the number of anxiety occurrences) as an index (step S158), and ends the processing.

As described above, the anxiety feature quantities can be calculated even in the case of the driving scene in which the anxiety is generally less likely to occur, and appropriate driving or appropriate advice as described later can be presented based on the calculated anxiety feature quantities.

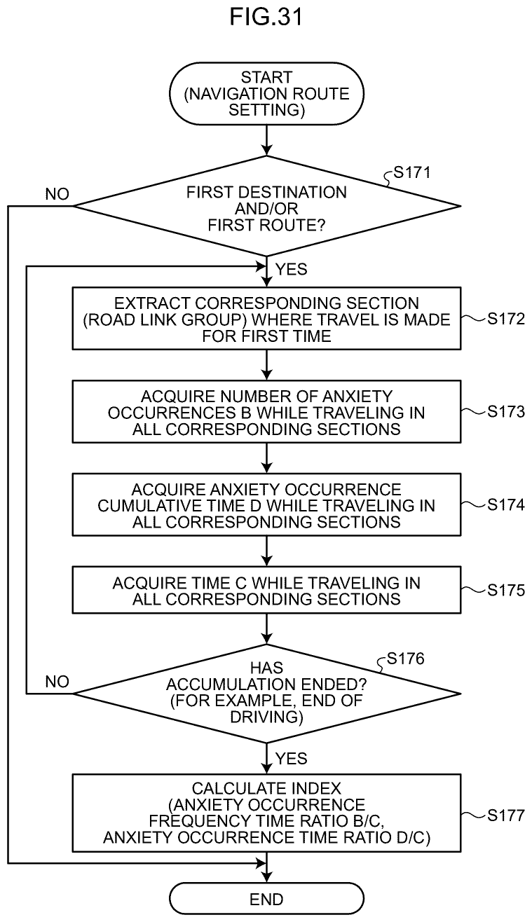

FIG. 31 is a processing flowchart of processing of calculating anxiety feature quantities on the first-time-driving road.

In this case, it is assumed that the counters (the anxiety occurrence number counter B, the driving time counter C for each first-time-driving road section, and the anxiety occurrence cumulative time counter D corresponding to each first-time-driving road) corresponding to each first-time-driving road section are respectively provided.

The anxiety feature calculation unit 53 resets the corresponding counter (the anxiety occurrence number counter B, the driving time counter C on the first-time-driving road section, and the anxiety occurrence cumulative time counter D corresponding to a first-time-driving road) prior to the processing of calculating anxiety feature quantities for each first-time-driving road.

First, when the route (navigation route) of the route guidance target is set, the anxiety feature calculation unit 53 determines whether or not the first destination and the first route are included or whether or not the first route is included, based on the driving history (step S171).

In the determination of step S171, when the first route is not included (step S171; No), the processing ends.

In the determination of step S171, when the route is a route including the first destination and the first route or a route including the first route (step S171; Yes), the anxiety feature calculation unit 53 extracts the corresponding section (road link group) where the vehicle travels for the first time (step 172).

Next, the anxiety feature calculation unit 53 acquires the number of anxiety occurrences during driving in all corresponding sections where the vehicle travels for the first time in the anxiety occurrence number counter B (step S173).

Subsequently, the anxiety feature calculation unit 53 acquires the anxiety occurrence cumulative time while traveling in all the corresponding sections where the vehicle travels for the first time in the anxiety occurrence cumulative time counter D (step S174).

Furthermore, the anxiety feature calculation unit 53 acquires the driving time while traveling in all the corresponding sections where the vehicle travels for the first time in the driving time counter C (step S175).

Next, the anxiety feature calculation unit 53 determines whether or not the accumulation end timing of the anxiety feature quantities has been reached, for example, whether or not the driving has ended (step S176).

In the determination of step S157, when the accumulation end timing of anxiety feature quantities has not been reached (step S176; No), the anxiety feature calculation unit 53 proceeds the processing to step S172 again, and thereafter, the above-described processing is executed.

In the determination of step S176, when the accumulation end timing of the anxiety feature quantities has been reached (step S176; Yes), the anxiety feature calculation unit 53 calculates the anxiety occurrence frequency time ratio (=the number of anxiety occurrences/the driving time) and the anxiety occurrence time ratio (=the anxiety occurrence cumulative time/the driving time) as an index (step S177), and ends the processing.

As described above, since the anxiety feature quantities can be calculated only in the section where the vehicle travels for the first time, matters to be noted when the vehicle travels in the section where the vehicle travels for the first time are also clarified, and appropriate driving can be performed even when the vehicle travels on an unfamiliar road, or appropriate advice can be presented as described later.

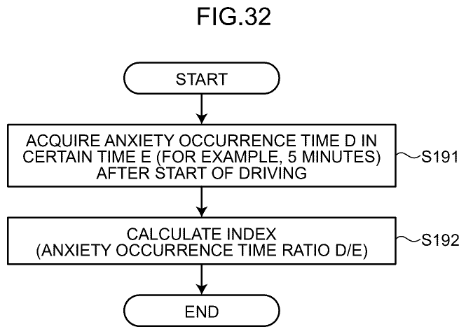

FIG. 32 is a processing flowchart of processing of calculating anxiety feature quantities in a predetermined period immediately after the start of driving.

Based on the output of the anxiety state acquisition unit 51, the anxiety feature calculation unit 53 acquires the anxiety occurrence time D in a certain time E (for example, 5 minutes) immediately after the start of driving (step S191).

Subsequently, the anxiety feature calculation unit 53 calculates the anxiety occurrence time ratio (=the anxiety occurrence time/certain time E) as an index (step S192), and ends the processing.

As described above, since the anxiety feature quantities can be calculated only for a certain time immediately after the start of driving, it is possible to carefully drive even in a time zone when the driver is not accustomed to driving immediately after the start of driving, or it is possible to present appropriate advice as described later.

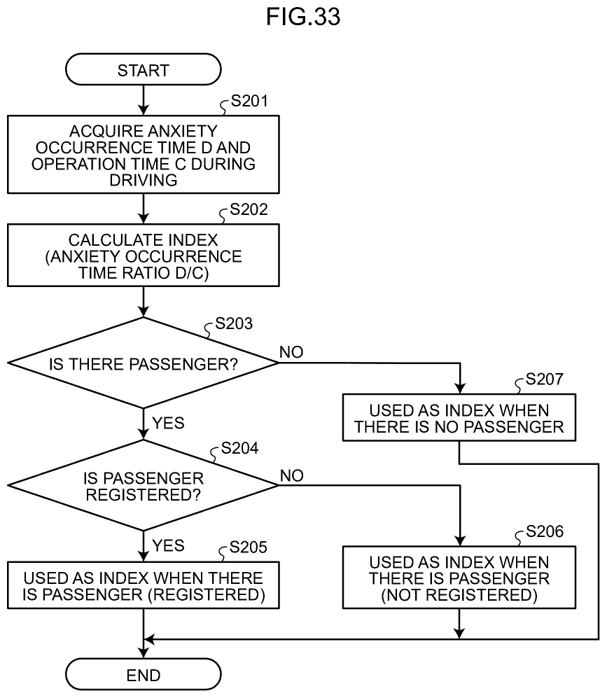

FIG. 33 is a processing flowchart of processing of calculating anxiety feature quantities in the presence or absence of a passenger.

This processing is performed after the end of driving.

In this case, it is assumed that corresponding counters (the anxiety occurrence cumulative time counter D and the driving time counter C) are provided during driving.

Based on the output of the anxiety state acquisition unit 51, the anxiety feature calculation unit 53 acquires the anxiety occurrence time during driving in the anxiety occurrence cumulative time counter D, and acquires the driving time (operation time) in the driving time counter C (step S201).

Subsequently, the anxiety feature calculation unit 53 calculates the anxiety occurrence time ratio (=the anxiety occurrence cumulative time/the driving time) as an index (step S202).

Next, the anxiety feature calculation unit 53 determines the presence or absence of a passenger based on the output of the passenger discrimination device using the image recognition function using the camera or the like (step S203). In this case, the passenger discrimination device can register a specific person (a person who frequently uses the vehicle, such as the person in question, a family member, an acquaintance, or an employee) as a registered passenger in advance according to an instruction of the user.

In the determination of step S203, when there is no passenger (step S203; No), the anxiety feature calculation unit 53 uses the index calculated in step S202 as an index in a case where there is no passenger (step S207), and ends the processing.

In the determination of step S203, when there is a passenger (step S203; Yes), the anxiety feature calculation unit 53 determines whether or not the passenger is a registered passenger based on the output of the passenger discrimination device (step S204).

In the determination of step S204, when the passenger is a registered passenger (step S204; Yes), the anxiety feature calculation unit 53 uses the index calculated in step S202 as an index in a case where the passenger is a registered passenger (step S205), and ends the processing.

In the determination of step S204, when the passenger is a registered passenger (step S204; No), the anxiety feature calculation unit 53 uses the index calculated in step S202 as an index in a case where there is a passenger but the passenger is not a registered passenger (step S206), and ends the processing.

As described above, since the index at the time of calculating the anxiety feature is selectively used based on the presence or absence of a passenger and further based on whether or not the passenger is a registered passenger, it is possible to calculate the anxiety feature quantities more accurately, to perform driving that more closely aligns with the psychological state of the driver during driving, or to present appropriate advice as described later.

As described above, according to the present fourth embodiment, it is possible to provide the anxiety feature quantities presentation device that accurately acquires the anxiety feature quantities more accurately and presents the anxiety feature quantities to the user based on the psychological state of the driver, the driving route, and the driving environment inside and outside the vehicle.

### (5) Fifth Embodiment

The present fifth embodiment relates to an information presentation device capable of presenting a change history of anxiety feature quantities to a user or presenting information such as advice during driving suitable for the user based on the history of the anxiety feature quantities.

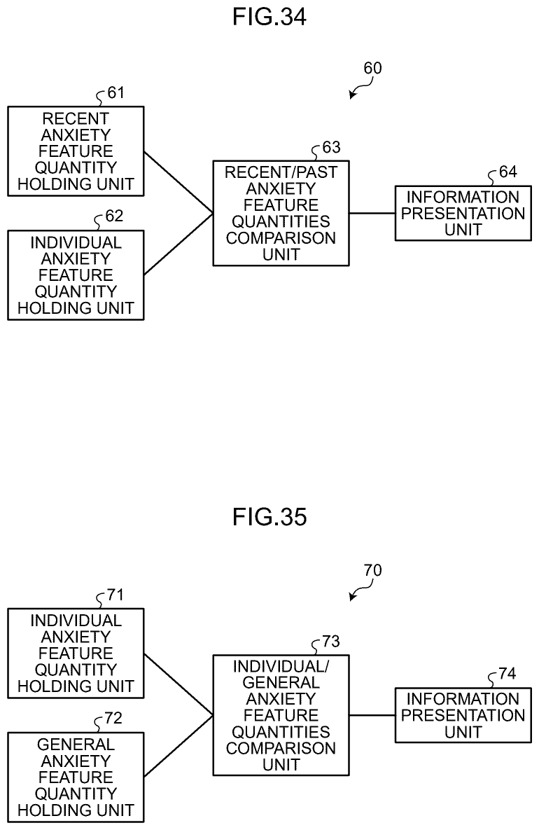

FIG. 34 is a functional configuration block diagram of an information presentation device according to a first aspect of the fifth embodiment.

The information presentation device 60 includes a recent anxiety feature quantity holding unit 61, an individual anxiety feature quantity holding unit 62, a recent/past anxiety feature quantities comparison unit 63, and an information presentation unit 64.

The recent anxiety feature quantity holding unit 61 holds the anxiety feature quantities of the user in the recent (latest) driving.

The individual anxiety feature quantity holding unit 62 holds the anxiety feature quantities of the user in the past driving.

The recent/past anxiety feature quantities comparison unit 63 compares the anxiety feature quantities of the user in the recent (latest) driving held by the recent anxiety feature quantity holding unit 61 with the anxiety feature quantities of the user in the past driving held by the individual anxiety feature quantity holding unit 62, and calculates the comparison result.

The information presentation unit 64 presents information based on the comparison result calculated by the recent/past anxiety feature quantities comparison unit 63.

The operation of the information presentation device 60 according to the first aspect will be described in detail later.

FIG. 35 is a functional configuration block diagram of an information presentation device according to a second aspect of the fifth embodiment.

The information presentation device 70 includes an individual anxiety feature quantity holding unit 71, a general anxiety feature quantity holding unit 72, an individual/general anxiety feature quantities comparison unit 73, and an information presentation unit 74.

The individual anxiety feature quantity holding unit 71 holds the anxiety feature quantities of the user in the past driving.

The general anxiety feature quantity holding unit 72 holds the anxiety feature quantities of an unspecified large number of users in the past driving.

The individual/general anxiety feature quantities comparison unit 73 compares the anxiety feature quantities of the user in the past driving held by the individual anxiety feature quantity holding unit 71 with the anxiety feature quantities of the unspecified large number of users in the past driving held by the general anxiety feature quantity holding unit 72, and calculates the comparison result.

The information presentation unit 74 presents information based on the comparison result calculated by the individual/general anxiety feature quantities comparison unit 73.

The operation of information presentation device 70 according to the second aspect will be described in detail later.

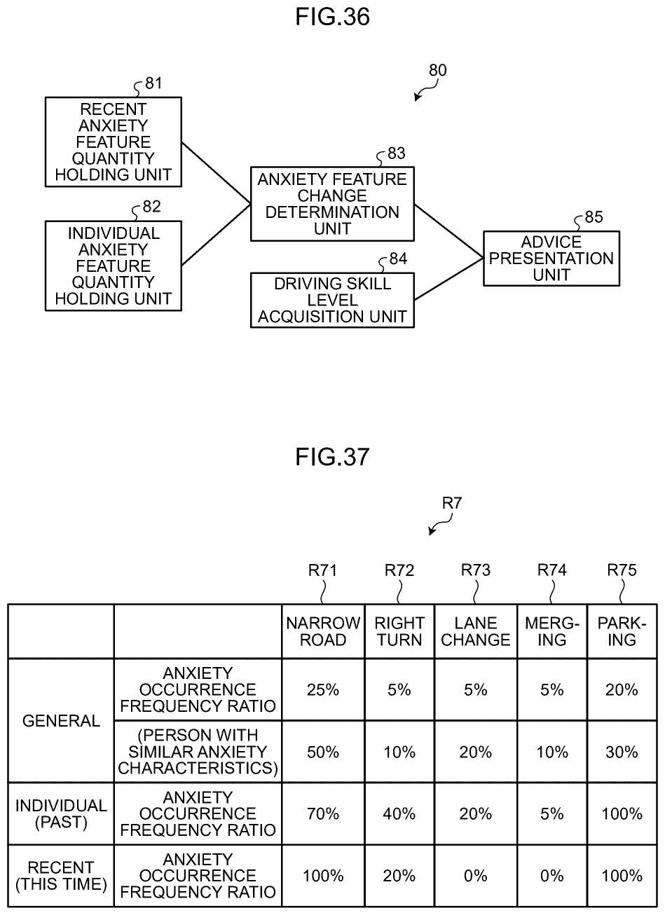

FIG. 36 is a functional configuration block diagram of an information presentation device according to a third aspect of the fifth embodiment.

An information presentation device 80 includes a recent anxiety feature quantity holding unit 81, an individual anxiety feature quantity holding unit 82, an anxiety feature change determination unit 83, a driving skill level acquisition unit 84, and an advice presentation unit 85.

The recent anxiety feature quantity holding unit 81 holds the anxiety feature quantities of the user in the recent (latest) driving.

The individual anxiety feature quantity holding unit 82 holds the anxiety feature quantities of the user in the past driving.

The anxiety feature change determination unit 83 compares the anxiety feature quantities of the user in the recent (latest) driving held by the recent anxiety feature quantity holding unit 61 with the anxiety feature quantities of the user in the past driving held by the individual anxiety feature quantity holding unit 62, determines the change in anxiety feature, and outputs the determination result to the advice presentation unit 85.

The driving skill level acquisition unit 84 acquires the driving skill level of the user and outputs an acquisition result to the advice presentation unit 85.

The advice presentation unit 85 presents advice regarding driving to the user based on the determination result of the change in the anxiety feature output by the anxiety feature change determination unit 83 and the driving skill level of the user acquired by the driving skill level acquisition unit 84.

The operation of the information presentation device 80 according to the third aspect will be described in detail later.

FIG. 37 is an explanatory diagram (part 1) of comparison data of anxiety feature quantities for each driving scene.

In the example of FIG. 37, a case of a narrow road, a right turn, a lane change, merging, and parking is illustrated as the driving scene.

As illustrated in FIG. 37, the anxiety feature quantities comparison result R7 includes the anxiety feature quantities comparison result R71 of a narrow road, the anxiety feature quantities comparison result R72 of a right turn, the anxiety feature quantities comparison result R73 of a lane change, the anxiety feature quantities comparison result R74 of merging, and the anxiety feature quantities comparison result R75 of parking.

Furthermore, FIG. 37 illustrates feature quantities (anxiety occurrence frequency ratio) of an unspecified large number of users, feature quantities (anxiety occurrence frequency ratio) of a large number of users having similar anxiety characteristics to the user who is a driver among the unspecified large number of users, feature quantities (anxiety occurrence frequency ratio) of the user in the past driving of the user, and feature quantities (anxiety occurrence frequency ratio) of the user in the recent driving of the user.

For example, in a right turn driving scene, the anxiety occurrence frequency ratio of the unspecified large number of users=5%, the anxiety occurrence frequency ratio for a large number of users having anxiety characteristics similar to those of the user=10%, the anxiety occurrence frequency ratio of the user in the past driving of the user=40%, and the anxiety occurrence frequency ratio of the user in the recent driving of the user=20%.

First, an operation example of the information presentation device 60 according to the first aspect will be described.

In the case of the example of FIG. 37, according to the information presentation device 60 according to the first aspect, the information presentation unit 64 presents, for example, the following information or advice to the user as information based on the comparison result calculated by the recent/past anxiety feature quantities comparison unit 63.

More specifically, the information presentation unit 64 of the information presentation device 60 presents a message of “This time, you were able to make right turns and lane changes calmly.”, for example, since the anxiety occurrence frequency ratio is improved in that the past anxiety occurrence frequency ratio at the time of turning right is 40%, whereas the recent anxiety occurrence frequency ratio at the time of turning right is 20%, and that the past anxiety occurrence frequency ratio at the time of lane change is 20%, whereas the recent anxiety occurrence frequency ratio at the time of lane change is 0%.

Furthermore, the information presentation unit 64 presents an advice of, for example, “Please check the technical advice separately for driving on narrow roads and parking.” from the viewpoint that the past anxiety occurrence frequency ratio on a narrow road was 70%, whereas the recent anxiety occurrence frequency ratio on a narrow road is 100%, and that the past anxiety occurrence frequency ratio at the time of parking was 100%, whereas the recent anxiety occurrence frequency ratio at the time of parking is 100%, showing no improvement at all.

Therefore, according to the information presentation device 60 according to the first aspect, it is possible to present appropriate information or advice to the current user based on the past anxiety occurrence situation of the user and the recent anxiety occurrence situation.

Next, an operation example of the information presentation device 70 according to the second aspect will be described.

In the case of the example of FIG. 37, according to the information presentation device 70 according to the second aspect, the information presentation unit 74 presents, for example, the following information or advice to the user as information based on the comparison result calculated by the individual/general anxiety feature quantities comparison unit 73.

More specifically, the information presentation unit 74 of the information presentation device 70 presents advice such as, for example, “It seems that driving on narrow roads, making right turns, and parking are causing some anxiety. Please check the technical advice separately.” based on the following points: the anxiety occurrence frequency ratio for general users on narrow roads is 25%, whereas the past anxiety occurrence frequency ratio for the individual user on narrow roads is 70%; the anxiety occurrence frequency ratio for general users when turning right is 5%, whereas the past anxiety occurrence frequency ratio for the individual user when turning right is 40%; and the anxiety occurrence frequency ratio for general users when parking is 20%, whereas the anxiety occurrence frequency ratio for the individual user when parking is 100%.

Furthermore, the information presentation unit 74 presents a message “You are able to merge calmly at the same level as others.”, for example, since the anxiety occurrence frequency ratio of the general user at the time of merging is 5% and the past anxiety occurrence frequency ratio of the individual user at the time of merging is also 5%, both are equivalent to each other.

Therefore, according to the information presentation device 70 according to the second aspect, it is possible to present appropriate information or advice to the user based on the past anxiety occurrence situation of the user and the anxiety occurrence situation of the unspecified large number of general users.

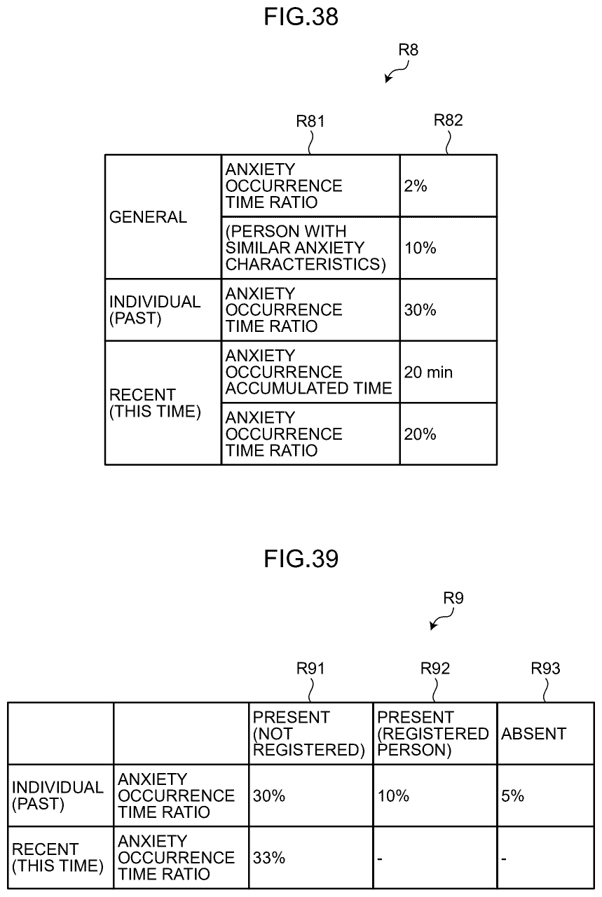

FIG. 38 is an explanatory diagram (part 2) of comparison data of anxiety feature quantities for each driving scene.

In the example of FIG. 38, a case of a first-time-driving road is illustrated as the driving scene.

As illustrated in FIG. 38, the anxiety feature quantities comparison result R8 includes a comparison result item R81 and anxiety feature quantities comparison result R82 of the first-time-driving road.

Furthermore, FIG. 38 illustrates feature quantities (anxiety occurrence frequency ratio) of an unspecified large number of users, feature quantities (anxiety occurrence frequency ratio) of a large number of users having similar anxiety characteristics to the user who is a driver among the unspecified large number of users, feature quantities (anxiety occurrence frequency ratio) of the user in the past driving of the user, and feature quantities (the anxiety occurrence accumulated time and the anxiety occurrence time ratio) of the user in the recent driving of the user.

In the driving scene of the first-time-driving road, in the case illustrated in FIG. 38, the anxiety occurrence time ratio of an unspecified large number of users =2%, the anxiety occurrence time ratio of a large number of users having similar anxiety characteristics to the user=10%, the anxiety occurrence time ratio of the user in the past driving of the user=30%, the anxiety occurrence accumulated time of the user in the recent driving of the user =20 minutes, and the anxiety occurrence time ratio of the user in the recent driving of the user=20%.

In the case of the example of FIG. 38, for example, advice of “When driving on a first-time-driving road, checking in advance the features and situations of the roads on the navigation route, as well as parking lots near the destination, can help you drive with greater peace of mind.” is presented from the viewpoint that the past anxiety occurrence time ratio of an individual user is 30% whereas the anxiety occurrence time ratio of a general user is 2%.

Furthermore, the message of “You were able to drive more calmly on a first-time-driving road than before. Keep up the confidence for your next drive.” is presented from the viewpoint that the past anxiety occurrence time ratio of the individual user is 30%, whereas the recent anxiety occurrence time ratio of the individual user is 20%.

Therefore, according to the information presentation device 70 according to the second aspect, it is possible to present appropriate information or advice to the user based on the past anxiety occurrence situation of the user and the anxiety occurrence situation of the unspecified large number of general users.

FIG. 39 is an explanatory diagram (part 3) of comparison data of anxiety feature quantities for each driving scene.

In the example of FIG. 39, a case of the presence or absence of a passenger is illustrated as the driving scene.

As illustrated in FIG. 39, the anxiety feature quantities comparison result R9 includes anxiety feature quantities comparison result R91 when there is a passenger (unregistered passenger), anxiety feature quantities comparison result R92 when there is a passenger (registered passenger), and anxiety feature quantities comparison result R93 when there is no passenger.

Furthermore, FIG. 39 illustrates the feature quantities (anxiety occurrence time ratio) of the user in the past driving of the user and the feature quantities (anxiety occurrence time ratio) of the user in the recent driving of the user.

In the driving scene related to the presence or absence of a passenger, in the case illustrated in FIG. 39, in the anxiety feature quantities comparison result R91 when there is a passenger (unregistered passenger), the anxiety occurrence time ratio of the user in the past driving of the user is 30%, whereas the anxiety occurrence time ratio of the user in the recent driving of the user is 33%, and further, in the anxiety feature quantities comparison result R92 when there is a passenger (registered passenger), the anxiety occurrence time ratio of the user in the past driving of the user is 10%. Therefore, an advice of “It seems that you become especially tense when driving with someone other than family. How about relaxing by listening to your favorite music while driving?” is presented.

Therefore, according to the information presentation device 70 according to the second aspect, it is possible to present appropriate information or advice to the user based on the past anxiety occurrence situation of the user and the anxiety occurrence situation of the unspecified large number of general users.

Next, an operation example of the information presentation device 80 according to the third aspect will be described.

Conventionally, driving skill has been evaluated from features of driving operation, behavior, and vehicle behavior, and advice according to the evaluation result has been given. However, since a change in ease of anxiety during driving (anxiety characteristics at the time of driving) is not reflected, appropriate advice has not been given in some cases.

More specifically, as a general tendency, the beginner driver improves the driving skill and reduces the anxiety at the time of driving as the driving experience is accumulated. On the other hand, the driving skill of the elderly driver gradually deteriorates due to the deterioration in ability, and the anxiety during driving increases.

In addition, there is a case where appropriate advice cannot be given only by the driving skill evaluation. That is, there is a case where the anxiety about driving is not changed even though the driving skill is improved, or a case where the anxiety about driving is not changed even though the driving skill deteriorates.

Therefore, the information presentation device 80 according to the third aspect operates to present appropriate advice even in the above-described case.

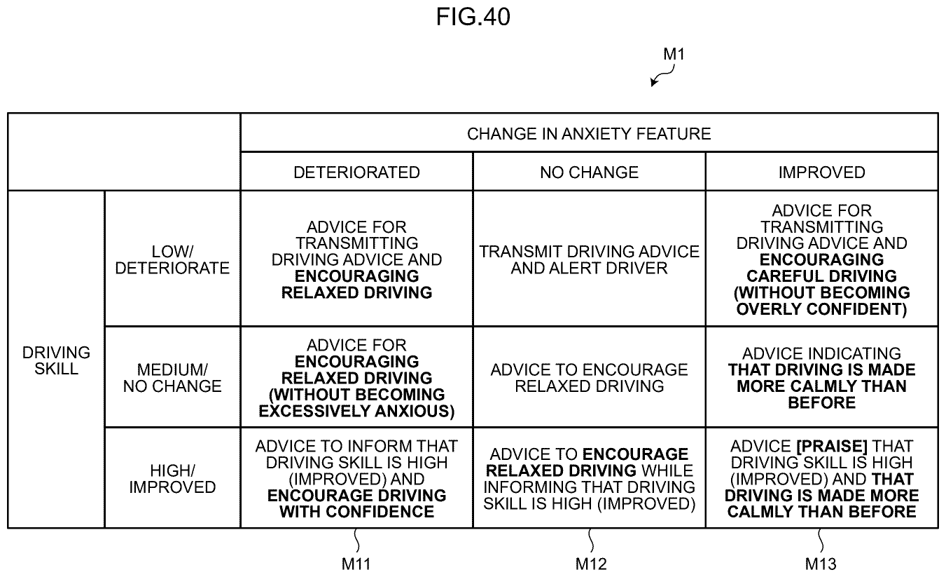

FIG. 40 is an explanatory diagram of an example of advice corresponding to a combination of driving skill and changes in anxiety feature.

An advice table M1 for a combination of a change in driving skill and an anxiety feature includes an advice group M11 in a case where the change in the anxiety feature deteriorates, an advice group M12 in a case where there is no change in the anxiety feature, and an advice group M13 in a case where the change in the anxiety feature is improved.

The information presentation device 80 includes the recent anxiety feature quantity holding unit 81, the individual anxiety feature quantity holding unit 82, the anxiety feature change determination unit 83, the driving skill level acquisition unit 84, and the advice presentation unit 85.

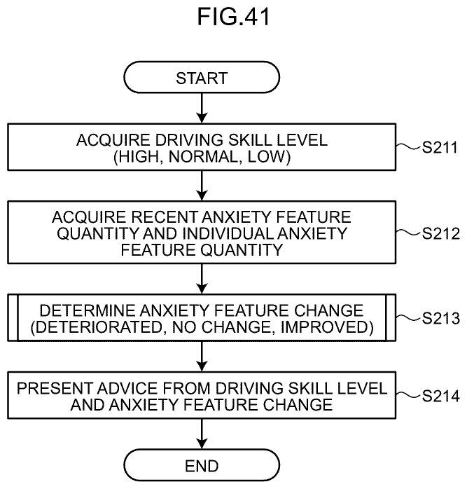

FIG. 41 is an operation flowchart of the information presentation device 80 according to the third aspect.

First, the driving skill level acquisition unit 84 of the information presentation device 80 acquires the driving skill level (high, normal, low) of the user (step S211).

Specifically, the driving skill level acquisition unit 84 identifies the driver who is the user by image recognition or the like, and compares the driving operation of the user and the vehicle behavior during driving with a predetermined model driving operation or a model vehicle behavior to acquire the driving skill level.

Next, the information presentation device 80 acquires the recent anxiety feature quantities held by the recent anxiety feature quantity holding unit 81 and the individual anxiety feature quantities held by the individual anxiety feature quantity holding unit 82 (step S212).

Then, the anxiety feature change determination unit 83 compares the recent anxiety feature quantities with the individual anxiety feature quantities, and determines whether or not the recent anxiety feature quantities deteriorate, are not changed, or are improved with respect to the individual anxiety feature quantities that is the past anxiety feature quantities (step S213).

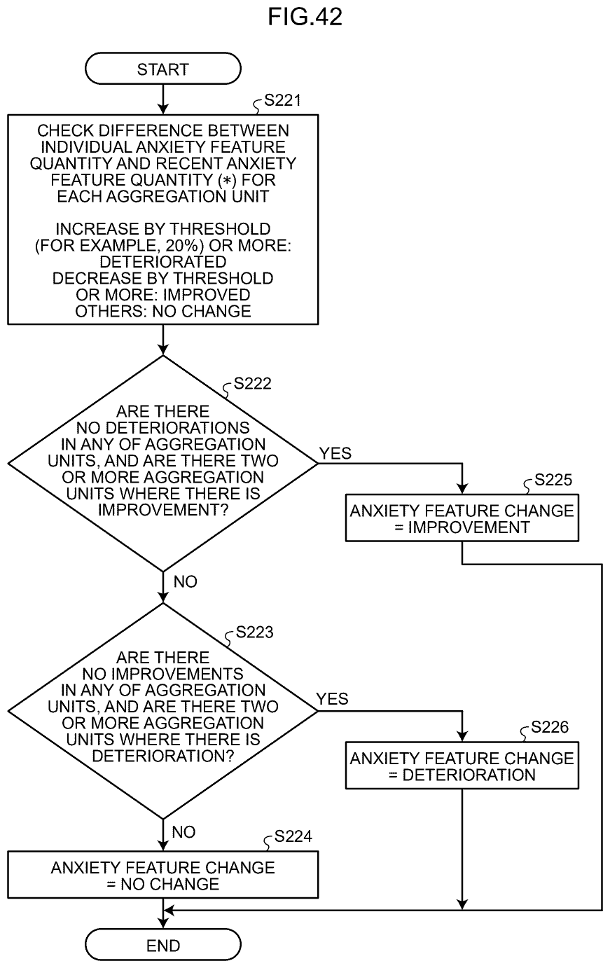

FIG. 42 is a processing flowchart of determination processing of an anxiety feature change determination unit.

First, the anxiety feature change determination unit 83 checks a difference between the recent anxiety feature quantities and the individual anxiety feature quantities acquired for each aggregation unit (step S221).

Specifically, for example, when the difference between the recent anxiety feature quantities and the individual anxiety feature quantities increases by a predetermined threshold (for example, 20%) or more, the anxiety feature change determination unit 83 determines that the recent anxiety feature quantities deteriorate with respect to the individual anxiety feature quantities which are the past anxiety feature quantities.

In addition, when the difference between the recent anxiety feature quantities and the individual anxiety feature quantities decreases by a predetermined threshold (for example, 20%) or more, the anxiety feature change determination unit 83 determines that the recent anxiety feature quantities deteriorate with respect to the individual anxiety feature quantities which are the past anxiety feature quantities. Furthermore, in other cases, the anxiety feature change determination unit 83 determines that there is no change.

Next, the anxiety feature change determination unit 83 determines whether or not the improvement determination condition is satisfied, for example, whether or not there is no deterioration in all the aggregation units and there is improvement in two or more aggregation units (step S222).

In the determination of step S222, when the improvement determination condition is satisfied (step S222; Yes), the anxiety feature change determination unit 83 determines that the anxiety feature change is improved (step S225), and ends the processing.

In the determination in step S222, when the improvement determination condition is not satisfied (step S222; No), the anxiety feature change determination unit 83 determines whether or not the deterioration determination condition is satisfied, for example, whether or not there is no improvement in all the aggregation units and there is deterioration in two or more aggregation units (step S223).

In the determination of step S223, when the deterioration determination condition is satisfied (step S223; Yes), the anxiety feature change determination unit 83 determines that the anxiety feature change deteriorates (step S226), and ends the processing.

In the determination of step S223, when the deterioration determination condition is not satisfied (step S223; No), the anxiety feature change determination unit 83 determines that there is no anxiety feature change (step S224), and ends the processing.

Then, the advice presentation unit 85 refers to the advice table M1 for the combination of the change in the driving skill and the change in the anxiety feature, and presents appropriate advice from the driving skill level and the anxiety feature change (step S214).

Specifically, in a case where the driving skill is high and improved and the anxiety feature change deteriorates, advice that conveys the message “to inform the user that their driving skill is high (or has improved) and to encourage them to drive with confidence” is given with reference to the advice group M11 in a case where the change in the anxiety feature deteriorates.

Furthermore, in a case where the driving skill is low or deteriorates and the anxiety feature change is improved, advice that conveys the message “to provide driving advice, and to encourage careful driving (without becoming overly confident)” corresponding to driving skill is given with reference to the advice group M13 in a case where the change in the anxiety feature is improved.

Furthermore, in a case where the driving skill is medium or has no change, and the anxiety feature change also has no change, advice that conveys the message “to encourage relaxed driving” is given with reference to the advice group M12 in a case where there is no change in the anxiety feature.

Therefore, according to the information presentation device 80 according to the third aspect, it is possible to present appropriate information or advice to the user based on the driving skill of the user and the change in the anxiety feature, and it is possible to encourage appropriate driving suitable for the driving skill.

While certain embodiments have been described, the above-described embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein can be made without departing from the spirit of the inventions. The accompanying claims and their equivalents are intended to cover such forms or modifications as would fall within the scope and spirit of the inventions.

Furthermore, the effects of the embodiments described in the present specification are merely examples and are not limited, and other effects may be provided.

The anxiety map display device, the information acquisition device, and the information presentation device of the embodiment have a hardware configuration using a normal computer including a control device such as an MPU, a storage device such as a read only memory (ROM) and a RAM, an input device, and an output device.

The program executed by the anxiety map display device, the information acquisition device, and the information presentation device of the present embodiment is provided by being recorded in a file in an installable format or an executable format in a semiconductor storage device such as a USB memory or an SSD, or a computer-readable recording medium such as a digital versatile disk (DVD).

In addition, the program executed by the anxiety map display device, the information acquisition device, and the information presentation device of the embodiment may be stored on a computer connected to a network such as the Internet and provided by being downloaded via the network. In addition, the program executed by the anxiety map display device, the information acquisition device, and the information presentation device of the present embodiment may be provided or distributed via a network such as the Internet.

In addition, the program of the anxiety map display device, the information acquisition device, and the information presentation device of the present embodiment may be provided by being incorporated in advance in a ROM or the like.

According to the present disclosure, it is possible to display an anxiety map or present anxiety feature quantities that provide information tailored to each driver based on the anxiety characteristics of each driver, thereby enabling driving with greater peace of mind.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

### Supplementary Notes

The present embodiment can also have the following aspects.

According to another first aspect, an anxiety map display device includes:
an anxiety state acquisition unit that acquires an anxiety state of a driver during driving;
a vehicle position acquisition unit that acquires position information of a vehicle during driving;
a driving situation acquisition unit that acquires a driving situation of the vehicle during driving;
an anxiety map generation unit that generates an anxiety map indicating an anxiety occurrence position corresponding to a predetermined driving situation for the driver based on the position information and the driving situation when a predetermined anxiety state is acquired for the driver by the anxiety state acquisition unit; and
an anxiety map display unit that displays the generated anxiety map for the driver as an individual anxiety map.

According to the above aspect, since the individual anxiety map reflecting the characteristics and attributes of the driver himself or herself can be displayed and used, it becomes possible to drive with a margin using the individual anxiety map based on the driving history up to that point.

In an anxiety map display device according to another second aspect, which is the anxiety map display device according to another first aspect,
a condition acquisition unit that acquires a condition to be displayed on the anxiety map from a user, is further provided, and
the anxiety map generation unit generates the anxiety map based on the acquired condition.

According to the above aspect, it is possible to display the individual anxiety map that matches the condition desired by the user, and convenience is improved.

In an anxiety map display device according to another third aspect, which is the anxiety map display device according to another second aspect,
the condition includes at least one of a narrow road, a right turn, a lane change, merging, parking, highway driving, nighttime driving, rainy weather driving, and a first-time-driving road.

According to the above aspect, it is possible to display the individual anxiety map of the condition under which the user feels anxiety more, and to easily use the individual anxiety map.

In an anxiety map display device according to another fourth aspect, which is the anxiety map display device according to another first aspect,
the anxiety map generation unit performs color coding based on the number of times the anxiety state is acquired at the same driving position and in the same driving situation.

According to the above aspect, it is possible to grasp the driving position that clearly becomes the anxiety state by color coding.

In an anxiety map display device according to another fifth aspect, which is the anxiety map display device according to another fourth aspect,
the anxiety map generation unit uses, as a display color, a color that is considered to be less likely to exacerbate anxiety in the color coding.

According to the above aspect, the anxiety map can be used without exacerbating the anxiety of the user by the display color.

In an anxiety map display device according to another sixth aspect, which is the anxiety map display device according to another fifth aspect,
a blue-based color and a green-based color are used as the display color, and use of a red-based color is prohibited.

According to the above aspect, depending on the display color, the anxiety map can be used in a calm state without increasing anxiety.

In an anxiety map display device according to another seventh aspect, which is the anxiety map display device according to another fifth aspect,
as the display color, a color that is likely to activate a parasympathetic nerve is used, and use of a color that is likely to activate a sympathetic nerve is prohibited.

According to the above aspect, by means of the display color, it is possible to use the anxiety map in a calm state without reliably increasing anxiety.

In an anxiety map display device according to another eighth aspect, which is the anxiety map display device according to another seventh aspect,
the color that is likely to activate a parasympathetic nerve includes a blue-based color and a green-based color,
a color that is likely to activate a sympathetic nerve includes a red-based color.

According to the above aspect, by means of the display color, it is possible to reliably calm the nerves and use the anxiety map without increasing anxiety.

In an anxiety map display device according to another ninth aspect, which is the anxiety map display device according to another first aspect,
the anxiety map generation unit excludes, from a target for generating the anxiety map, the position information and the driving situation acquired in a period in which a predetermined specific condition is satisfied.

According to the above aspect, the reliability of the anxiety map can be improved.

In an anxiety map display device according to another tenth aspect, which is the anxiety map display device according to another ninth aspect,
the specific condition is at least one of when acquired before a predetermined time elapses after the vehicle has started, when acquired before a moving distance after the vehicle has started exceeds a predetermined distance, when acquired from a predetermined time before arrival at a destination until arrival at the destination, when acquired after approaching the destination by a predetermined distance until arrival at the destination, and when there is a passenger.

According to the above aspect, since the information on the anxiety state collected in the low reliability state is not reflected in the anxiety map, the reliability of the anxiety map can be improved.

An anxiety map display device according to another eleventh aspect includes:
an anxiety state acquisition unit that acquires an anxiety state of a driver during driving;
a vehicle position acquisition unit that acquires position information of a vehicle during driving;
an anxiety map generation unit that generates an anxiety map indicating an anxiety occurrence position for the driver based on the position information when a predetermined anxiety state is acquired for the driver by the anxiety state acquisition unit; and
an anxiety map display unit that displays the generated anxiety map for the driver as an individual anxiety map.

According to the above aspect, since the individual anxiety map reflecting the characteristics and attributes of the driver himself or herself can be displayed and used, it becomes possible to drive with a margin using the individual anxiety map based on the driving history up to that point.

An anxiety map display device according to another twelfth aspect includes:
a plurality of information acquisition devices each mounted on a vehicle, the information acquisition devices each including an anxiety state acquisition unit that acquires an anxiety state of a driver during driving, a vehicle position acquisition unit that acquires position information of a vehicle during driving, a driving situation acquisition unit that acquires a driving situation of the vehicle during driving, and a driver characteristics/attributes acquisition unit that acquires characteristics and attributes of the driver;
a general anxiety map generation unit that generates a general anxiety map indicating an anxiety occurrence position corresponding to a predetermined driving situation for a plurality of drivers based on the position information and the driving situation when a predetermined anxiety state is acquired for the driver by the anxiety state acquisition unit; and
an anxiety map display unit that displays the generated anxiety map.

According to the above aspect, since the statistical general anxiety map reflecting the characteristics and attributes of the plurality of general drivers can be displayed and used, it becomes possible to drive with a margin using the general anxiety map reflecting the anxiety situation of the general drivers.

In an anxiety map display device according to another thirteenth aspect, which is the anxiety map display device according to another twelfth aspect,
a condition acquisition unit that acquires a condition to be displayed on the general anxiety map, is further provided, and
the general anxiety map generation unit generates the general anxiety map based on the acquired condition.

According to the above aspect, it is possible to display the general anxiety map that matches the condition desired by the user, and convenience is improved.

In an anxiety map display device according to another fourteenth aspect, which is the anxiety map display device according to another thirteenth aspect,
the condition includes at least one of a narrow road, a right turn, a lane change, merging, parking, highway driving, nighttime driving, rainy weather driving, and a first-time-driving road.

According to the above aspect, it is possible to display the general anxiety map of the condition under which the user feels anxiety more, and to easily use the general anxiety map.

In an anxiety map display device according to another fifteenth aspect, which is the anxiety map display device according to another twelfth aspect,
the general anxiety map generation unit performs color coding based on the number of times the anxiety state is acquired at the same driving position and in the same driving situation.

According to the above aspect, the anxiety map can be used without exacerbating the anxiety of the user by the display color.

An anxiety feature presentation device according to another sixteenth aspect includes:
an anxiety state acquisition unit that acquires an anxiety state of a driver during driving;
a vehicle position acquisition unit that acquires position information of a vehicle during driving;
a driving situation acquisition unit that acquires a driving situation of the vehicle during driving;
an anxiety feature calculation unit that calculates an anxiety feature quantity for each predetermined aggregation unit, based on the position information and the driving situation when a predetermined anxiety state is acquired for the driver by the anxiety state acquisition unit; and
an anxiety feature presentation unit that presents the calculated an anxiety feature quantity.

According to the above aspect, it is possible to reliably present the anxiety feature quantity of the driver himself or herself and to drive with a margin.

In an anxiety feature presentation device according to another seventeenth aspect, which is the anxiety feature presentation device according to another sixteenth aspect,
the driving situation includes at least one of a narrow road, a right turn, a lane change, merging, parking, highway driving, nighttime driving, rainy weather driving, and a first-time-driving road.

According to the above aspect, it is possible to present the anxiety feature of the condition under which the user feels anxiety more, and to easily use the anxiety feature.

In an anxiety feature presentation device according to another eighteenth aspect, which is the anxiety feature presentation device according to another sixteenth aspect,
the anxiety feature quantity includes at least one of the number of anxiety occurrences, anxiety occurrence cumulative time, anxiety occurrence frequency ratio, an anxiety occurrence time ratio, and the number of anxiety occurrences per unit time.

According to the above aspect, it is possible to present the anxiety feature of the condition under which the user feels anxiety more, and to easily use the anxiety feature.

In an anxiety feature presentation device according to another nineteenth aspect, which is the anxiety feature presentation device according to another sixteenth aspect,
the aggregation unit is any one of a driving scene, a driving environment, a driving scene in which anxiety is generally considered to be less likely to occur, presence or absence of a passenger, a first-time-driving road, and a predetermined period immediately after the start of driving.

According to the above aspect, it is possible to present the anxiety feature of the condition under which the driver feels anxiety more, and to easily use the anxiety feature.

In an anxiety feature presentation device another twentieth aspect, which is the anxiety feature presentation device according to another sixteenth to nineteenth aspects,
an advice presentation unit that presents advice based on a result of comparison between the anxiety feature quantities of the same aggregation unit, is further provided.

According to the above aspect, it is possible to present a more suitable advice in consideration of the result of comparison of the anxiety feature quantities of the same aggregation unit, that is, the change state of the anxiety feature quantities.

In an anxiety feature presentation device according to another twenty first aspect, which is the anxiety feature presentation device according to another twentieth aspect,
the advice presentation unit presents the advice for the anxiety feature quantities of the same aggregation unit based on the result of comparison between a past anxiety feature quantity and a recent anxiety feature quantity.

According to the above aspect, it is possible to present advice in consideration of not only the recent anxiety feature quantity but also the process of changing from the past anxiety feature quantity to the recent anxiety feature quantity, and thus, it is possible to guide the user to a more meaningful direction.

An anxiety feature presentation device according to another twenty second aspect, which is the anxiety feature presentation device according to another sixteenth aspect, further includes:
an anxiety feature change determination unit that determines a temporal change in the anxiety feature quantity;
a driving skill level acquisition unit that acquires driving skill information; and
an advice presentation unit that presents advice based on a relationship between the temporal change in the anxiety feature quantity and the driving skill.

According to the above aspect, since advice suitable for the driving skill of the driver and the anxiety state of the driver can be presented, driving can be performed in a more relieved state.

What is claimed is:

1. An anxiety map display device comprising:
a memory; and
a processor coupled to the memory, and configured to:
acquire an anxiety state of a driver during driving;
acquire position information of a vehicle during driving;
acquire a driving situation of the vehicle during driving;
generate an anxiety map indicating an anxiety occurrence position corresponding to a predetermined driving situation for the driver based on the position information and the driving situation when acquiring a predetermined anxiety state for the driver; and
display the generated anxiety map for the driver as an individual anxiety map.

2. The anxiety map display device according to claim 1, wherein
the processor is further configured to acquire a condition to be displayed on the anxiety map from a user, and
the processor is configured to generate the anxiety map based on the acquired condition.

3. The anxiety map display device according to claim 2, wherein
the condition includes at least one of a narrow road, a right turn, a lane change, merging, parking, highway driving, nighttime driving, rainy weather driving, and a first-time-driving road.

4. The anxiety map display device according to claim 1, wherein
the processor is configured to perform color coding based on a number of times the anxiety state is acquired at the same driving position and in the same driving situation.

5. The anxiety map display device according to claim 4, wherein
the processor is configured to use, as a display color, a color that is considered to be less likely to exacerbate anxiety in the color coding.

6. The anxiety map display device according to claim 5, wherein
as the display color, a blue-based color and a green-based color are used, and use of a red-based color is prohibited.

7. The anxiety map display device according to claim 5, wherein
as the display color, a color that is likely to activate a parasympathetic nerve is used, and use of a color that is likely to activate a sympathetic nerve is prohibited.

8. The anxiety map display device according to claim 7, wherein
the color that is likely to activate the parasympathetic nerve includes a blue-based color and a green-based color, and
a color that is likely to activate the sympathetic nerve includes a red-based color.

9. The anxiety map display device according to claim 1, wherein
the processor is configured to exclude, from a target for generating the anxiety map, the position information and the driving situation acquired in a period in which a predetermined specific condition is satisfied.

10. The anxiety map display device according to claim 9, wherein
the specific condition is at least one of when acquired before a predetermined time elapses after the vehicle has started, when acquired before a moving distance after the vehicle has started exceeds a predetermined distance, when acquired from a predetermined time before arrival at a destination until arrival at the destination, when acquired after approaching the destination to a predetermined distance until arrival at the destination, and when there is a passenger.

11. An anxiety map display device comprising:
a plurality of information acquisition devices each mounted on a vehicle, the plurality of information acquisition devices each including: a first memory; and a first processor coupled to the first memory and configured to: acquire an anxiety state of a driver during driving; acquire position information of a vehicle during driving; acquire a driving situation of the vehicle during driving; and acquire characteristics and attributes of the driver;
a second memory; and
a second processor couplet to the second memory, and configured to:
generate a general anxiety map indicating an anxiety occurrence position corresponding to a predetermined driving situation for a plurality of drivers based on the position information and the driving situation when acquiring a predetermined anxiety state for the driver; and
display the generated general anxiety map.

12. The anxiety map display device according to claim 11, wherein
the second processor is further configured to acquire a condition to be displayed on the general anxiety map, and
the second processor is configured to generate the general anxiety map based on the acquired condition.

13. The anxiety map display device according to claim 12, wherein
the condition includes at least one of a narrow road, a right turn, a lane change, merging, parking, highway driving, nighttime driving, rainy weather driving, and a first-time-driving road.

14. An anxiety feature presentation device comprising:
a memory; and
a processor couplet to the memory and configured to:
acquire an anxiety state of a driver during driving;
acquire position information of a vehicle during driving;
acquire a driving situation of the vehicle during driving;
calculate an anxiety feature quantity for each predetermined aggregation unit, based on the position information and the driving situation when a predetermined anxiety state is acquired for the driver by the anxiety state acquisition unit; and
present the calculated anxiety feature quantity.

15. The anxiety feature presentation device according to claim 14, wherein
the driving situation includes at least one of a narrow road, a right turn, a lane change, merging, parking, highway driving, nighttime driving, rainy weather driving, and a first-time-driving road.

16. The anxiety feature presentation device according to claim 14, wherein
the anxiety feature quantity includes at least one of a number of anxiety occurrences, anxiety occurrence cumulative time, anxiety occurrence frequency ratio, an anxiety occurrence time ratio, and a number of anxiety occurrences per unit time.

17. The anxiety feature presentation device according to claim 14, wherein
the aggregation unit is any one of a driving scene, a driving environment, a driving scene in which anxiety is generally considered to be less likely to occur, presence or absence of a passenger, a first-time-driving road, and a predetermined period immediately after a start of driving.

18. The anxiety feature presentation device according to claim 14, wherein
the processor is further configured to present advice based on a result of comparison between anxiety feature quantities of the same aggregation unit.

19. The anxiety feature presentation device according to claim 18, wherein
the processor is configured to present the advice for the anxiety feature quantities of the same aggregation unit based on the result of comparison between a past anxiety feature quantity and a recent anxiety feature quantity.

20. The anxiety feature presentation device according to claim 14, wherein
the processor is further configured to:
determine a temporal change in the anxiety feature quantity;
acquire driving skill information; and
present advice based on a relationship between the temporal change in the anxiety feature quantity and the driving skill.
